(19) United States
(12) Patent Application Publication
(10) Pub. No.: US 2023/0366020 A1
(43) Pub. Date: Nov. 16, 2023

(54) USE OF UNIQUE MOLECULAR IDENTIFIERS FOR IMPROVED ACCURACY OF LONG READ SEQUENCING AND CHARACTERIZATION OF CRISPR EDITING

(71) Applicant: INTEGRATED DNA TECHNOLOGIES, INC., Coralville, IA (US)

(72) Inventors: Mollie Schubert, Cedar Rapids, IA (US); Thomas Osborne, Iowa City, IA (US); Matthew McNeill, Coralville, IA (US); Garrett Rettig, Coralville, IA (US)

(21) Appl. No.: 18/316,454

(22) Filed: May 12, 2023

Related U.S. Application Data
(60) Provisional application No. 63/341,850, filed on May 13, 2022.

(51) Int. Cl. C12Q 1/6869; C12Q 1/6851

(52) U.S. Cl. CPC C12Q 1/6869; C12Q 1/6851; C12Q 1/6806

(57) ABSTRACT

Described herein is a system and process for long read sequencing using PCR primers with incorporated Unique Molecular Identifiers (UMIs) for generating a single molecule consensus for each starting molecule in the sample population. This method reduces the sequencing error rate by generating a consensus from the individual reads in each UMI group, averaging out sequencing errors to give better confidence in the actual sequence, to allow for increased accuracy of quantifying the precise knock-in event, and reporting perfect HDR integration.

### CROSS-REFERENCE TO RELATED APPLICATIONS

This application claims priority to U.S. Provisional Patent Application No. 63/341,850, filed May 13, 2022, which is incorporated by reference herein in its entirety. This application is related to U.S. patent application Ser. No. 16/919,577 and International Patent Application No. PCT/US2020/040621, both filed on Jul. 2, 2020, each of which is incorporated by reference herein in its entirety.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “013670-9069-US02_sequence_listing_xml_28-APR-2023.xml,” was created on May 1, 2023, contains 26 sequences, has a file size of 48.8 Kbytes, and is incorporated by reference in its entirety into the specification.

### TECHNICAL FIELD

Described herein is a system and process for long read sequencing using polymerase chain reaction (PCR) primers with incorporated Unique Molecular Identifiers (UMIs) for generating a single molecule consensus for each starting molecule in the sample population. This method reduces the sequencing error rate by generating a consensus from the individual reads in each UMI group, averaging out sequencing errors to give better confidence in the actual sequence, to allow for increased accuracy of quantifying the precise knock-in event, and reporting perfect homology-directed repair (HDR) integration.

### BACKGROUND

Long read sequencing using Oxford Nanopore Technologies (ONT) and/or PacBio sequencing platforms is advantageous over existing methods for characterizing CRISPR-Cas9 homology-directed repair (HDR) mediated knock-in because it provides phased information across the entirety of the edited genomic locus including the knocked-in sequence. This allows for the confirmation that the exogenous sequence of interest has been integrated as intended. However, long read sequencing using currently available platforms is highly prone to sequencing errors, which limits the utility of these systems for accurate base by base resolution of a knock-in sequence in a highly diverse polyclonal population. There is often insufficient edited genomic DNA to use amplification-free enrichment strategies (e.g., target enrichment) so PCR is required to generate sufficient material for sequencing. However, during library preparation, PCR amplification from a genomic DNA sample can result in biased representation of the wild-type (WT) and HDR sequences in the final sequencing library due to the difference in amplification efficiency between the shorter WT sequence and longer knock-in containing sequence. This “PCR bias” can artificially decrease the measured HDR frequency, leading to an underrepresentation of the actual knock-in integration efficiency.

What is needed is an algorithm and process (together forming new methods) for the improved accuracy of long read sequencing and characterization of CRISPR editing. These methods would be useful for characterizing the performance of CRISPR-Cas9 HDR mediated knock-in applications.

### SUMMARY

One embodiment described herein is a method for improving the accuracy of long read sequencing, the method comprising: generating a sequencing library comprising: (a) amplifying a locus with primers comprising a unique molecular identifier and a universal sequence to generate an initial product; (b) purifying the initial products; (c) amplifying the initial product with primers comprising a sequence complementary to the universal sequence and a barcode sequence to generate barcoded products; (d) purifying the barcoded products to produce purified barcoded products; (e) pooling the purified barcoded products to produce pooled barcoded products; and (f) sequencing the pooled barcoded products using a long-read sequencing apparatus to generate raw nucleotide sequence data. In one aspect the method further comprises, executing on a processor: (g) receiving raw nucleotide sequence data; (h) aligning the raw nucleotide sequence data to a reference amplicon to generate mapped sequences; (i) identifying and separating mapped sequences by target regions to generate a plurality of groups of target region sequences; (j) for each group of target region sequences: (i) analyzing the target region sequences for unique molecular identifiers and discarding target region sequences lacking a unique molecular identifier; (ii) clustering target region sequences containing unique molecular identifiers to generate clustered target region sequences and a cluster consensus sequence; (iii) analyzing and filtering the clustered target region sequences and discarding sequences with less than an elected number of cluster consensus sequences and downsampling clusters with greater than an elected cluster size to the elected cluster size; (iv) generating an inital target sequence consensus sequence; (k) repeating steps (j) on the inital target sequence consensus sequences to create a high accuracy consensus sequence for each cluster group, and correct amplification bias by clustering groups that were not similar enough to be clustered in the first round; (I) outputting high accuracy consensus sequence data. In another aspect, step (j)(i) comprises: aligning 5′- and 3′-adapters and UMI-adjacent substrings of the target region to both end substrings of the sequences; nucleotides between the aligned target sequence and adapter sequence on each end identify and enable clustering of the UMI sequences; and sequences lacking UMIs at both ends and containing less than 3 edit differences to the UMI are discarded. In another aspect, the elected number of cluster consensus sequences is between 3 and 10; and the elected cluster size is 20 to 80. In another aspect, the method further comprises analyzing the raw nucleotide sequence data from step 1(f) or the high accuracy consensus sequence data from step 2(1), comprising, executing on a processor: receiving the sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics. In another aspect, purifying in steps (b) and (d) comprises solid phase reversible immobilization (SPRI) purification. In another aspect, the unique molecular identifier comprises 8-30 nucleotides. In another aspect, the unique molecular identifier comprises 8-18 nucleotides. In another aspect, the universal sequence comprises 22-30 nucleotides. In another aspect, the barcode sequence comprises 16-24 nucleotides. In another aspect, the amplifying in step (a) comprises at least 2 cycles of PCR. In another aspect, the amplifying in step (a) comprises 2-4 cycles of PCR. In another aspect, the amplifying in step (c) comprises 20-40 cycles of PCR. In another aspect, long-read sequencing apparatus are selected from Oxford Nanopore Technologies (ONT) MinION, or PacBio Sequel II. In another aspect, the sequencing error rate is reduced by at least 15-fold.

### DESCRIPTION OF THE DRAWINGS

FIG. 1 shows the library preparation workflow. Two cycles of PCR with UMI containing primers were used to incorporate unique UMIs on each end of each input DNA molecule. The reaction was subsequently amplified using universal tails to generate multiple copies of each UMI group for sequencing.

FIG. 2 shows the modified UMI consensus construction workflow from Oxford Nanopore Technologies' github.

FIG. 3A-3B show the fraction of HDR reads that are considered perfect in CRISPAltRations output without UMI consensus construction (raw) or with UMI consensus construction using the min10 or min3 parameters. FIG. 3A shows results from sequencing using Oxford Nanopore Technologies' MinION device (R9.4.1 chemistry, Kit9). FIG. 3B shows results from sequencing on PacBio Sequel II device (HiFi CCS reads, min20 Q score).

FIG. 4 shows CRISPAltRations HDR quantification without UMI consensus construction (raw) or with UMI consensus construction using either a UMI sequence similarity of 80% and a minimum intermediate cluster size of 3 or 10. The expected percent HDR for the sample is plotted in heavy pixellation.

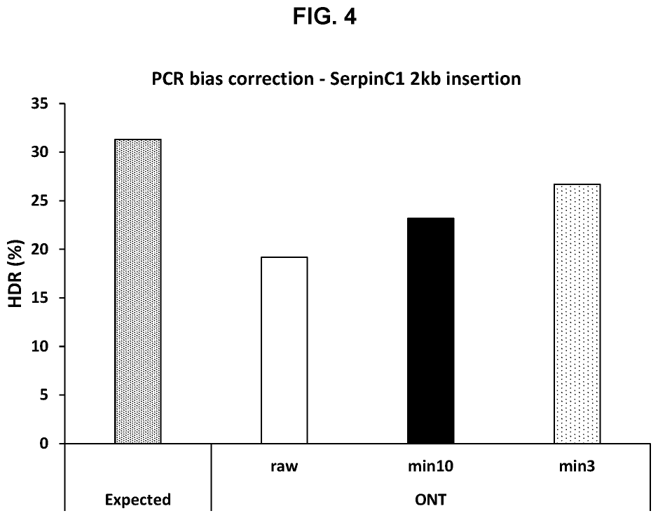

FIG. 5 shows the general workflow for CRISPAltRations for short and long read sequences. Edited genomic DNA is extracted and amplified using targeted multiplex PCR to enrich for the on- and predicted off-target loci. Amplicons are sequenced on an Illumina MiSeq. Read pairs are merged into a single fragment (FLASH), mapped to the genome (minimap2), and binned by their alignment to expected amplicon positions. Reads in each bin are re-aligned to the expected amplicon sequence after finding the cut site and creating a position specific gap open/extension bonus matrix to preferentially align indels closer to the cut site/expected indel profiles for each enzyme (CRISPAltRations code+psnw). Indels that intersected with a window upstream or downstream of the cut site were annotated. Percent editing is the sum of reads containing indels/total observed.

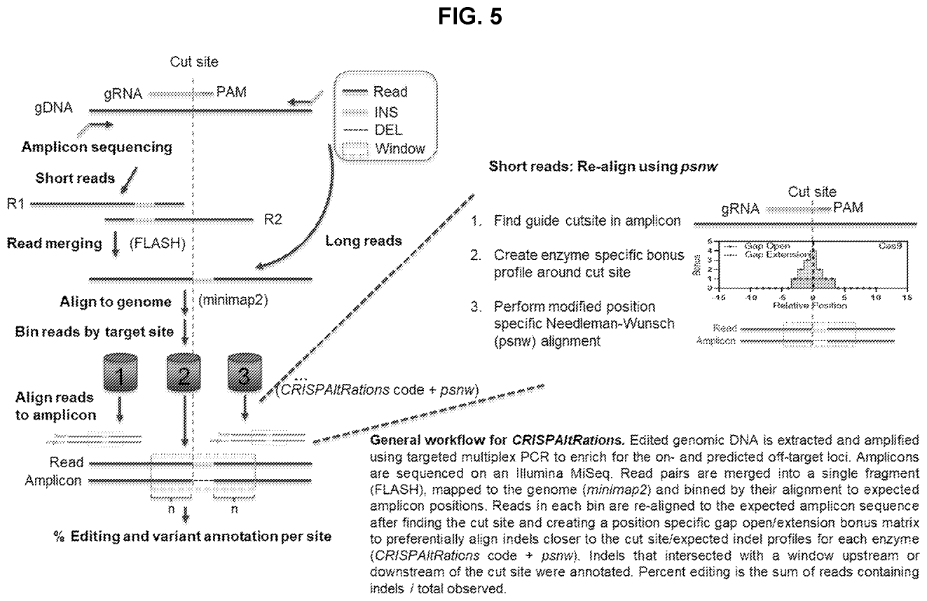

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. As used herein in nucleotide sequences, “N” refers to any nucleotide, e.g., A, T, C, G; “R” refers to purine nucleotides, e.g., C or G; and “Y” refers to pyrimidine nucleotides, e.g., A or T. Some nucleotide sequences have a 5′-amino-C₆ modification, e.g., 5′-NH₂(CH₂)₆PO₄—, which is abbreviated “/5AmMC6/.”

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments.

Described herein is the use of Unique Molecular Identifiers (UMIs) incorporated within PCR primers to allow for generation of a single molecule consensus for each starting molecule in the sample population. This method can correct for PCR bias and allows for a more accurate count of the number of starting molecules before PCR amplification. By extension, for this application, consolidation of reads by matched UMIs enables better quantification of the HDR frequency. Additionally, this method reduces the sequencing error rate by generating a consensus from the individual reads in each UMI group, averaging out sequencing errors to give better confidence in the actual sequence, to allow for increased accuracy of quantifying the precise knock-in event, and reporting perfect HDR integration.

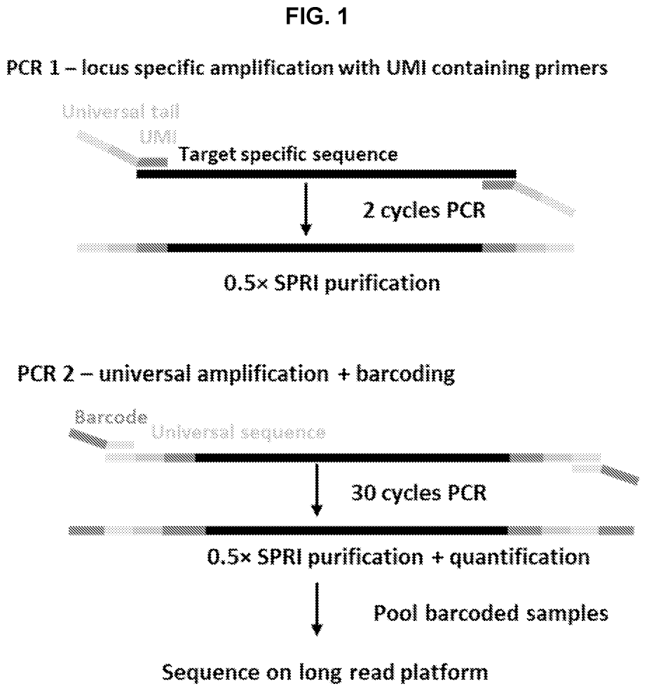

In this method, PCR primers are designed to include a target-specific sequence, a UMI, and a universal 5′-end for a secondary barcoding step to allow for sample multiplexing on a sequencing run (see FIG. 1). After sequencing, basecalling, and demultiplexing, multiple sequencing reads for each UMI are used to construct a consensus sequence via a bioinformatic pipeline. The final UMI consensus sequences can be used for downstream analysis, such as CRISPR editing characterization and HDR quantification using the CRISPAltRations analysis pipeline (Integrated DNA Technologies Inc.) as described in U.S. Patent Application Publication No. US 2021/0002700 A1 and International Patent Application Publication No. WO 2021/00 3343 A1, each of which are incorporated by reference herein for their teachings.

One embodiment described herein is a method for improving the accuracy of long read sequencing, the method comprising: generating a sequencing library comprising: (a) amplifying a locus with primers comprising a unique molecular identifier and a universal sequence to generate an initial product; (b) purifying the initial products; (c) amplifying the initial product with primers comprising a sequence complementary to the universal sequence and a barcode sequence to generate barcoded products; (d) purifying the barcoded products to produce purified barcoded products; (e) pooling the purified barcoded products to produce pooled barcoded products; and (f) sequencing the pooled barcoded products using a long-read sequencing apparatus to generate raw nucleotide sequence data. In one aspect the method further comprises, executing on a processor: (g) receiving raw nucleotide sequence data; (h) aligning the raw nucleotide sequence data to a reference amplicon to generate mapped sequences; (i) identifying and separating mapped sequences by target regions to generate a plurality of groups of target region sequences; (j) for each group of target region sequences: (i) analyzing the target region sequences for unique molecular identifiers and discarding target region sequences lacking a unique molecular identifier; (ii) clustering target region sequences containing unique molecular identifiers to generate clustered target region sequences and a cluster consensus sequence; (iii) analyzing and filtering the clustered target region sequences and discarding sequences with less than an elected number of cluster consensus sequences and downsampling clusters with greater than an elected cluster size to the elected cluster size; (iv) generating an inital target sequence consensus sequence; (k) repeating steps (j) on the inital target sequence consensus sequences to create a high accuracy consensus sequence for each cluster group, and correct amplification bias by clustering groups that were not similar enough to be clustered in the first round; (I) outputting high accuracy consensus sequence data. In another aspect, step (j)(i) comprises: aligning 5′- and 3′-adapters and UMI-adjacent substrings of the target region to both end substrings of the sequences; nucleotides between the aligned target sequence and adapter sequence on each end identify and enable clustering of the UMI sequences; and sequences lacking UMIs at both ends and containing less than 3 edit differences to the UMI are discarded. In another aspect, the elected number of cluster consensus sequences is between 3 and 10; and the elected cluster size is 20 to 80. In another aspect, the method further comprises analyzing the raw nucleotide sequence data from step 1(f) or the high accuracy consensus sequence data from step 2(l), comprising, executing on a processor: receiving the sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics. In another aspect, purifying in steps (b) and (d) comprises solid phase reversible immobilization (SPRI) purification. In another aspect, the unique molecular identifier comprises 8-30 nucleotides. In another aspect, the unique molecular identifier comprises 8-18 nucleotides. In another aspect, the universal sequence comprises 22-30 nucleotides. In another aspect, the barcode sequence comprises 16-24 nucleotides. In another aspect, the amplifying in step (a) comprises at least 2 cycles of PCR. In another aspect, the amplifying in step (a) comprises 2-4 cycles of PCR. In another aspect, the amplifying in step (c) comprises 20-40 cycles of PCR. In another aspect, long-read sequencing apparatus are selected from Oxford Nanopore Technologies (ONT) MinION, or PacBio Sequel II. In another aspect, the sequencing error rate is reduced by at least 15-fold.

Another embodiment described herein is an analytical pipeline called CRISPAltRations. This pipeline typically takes in FASTQ files and builds a merged R₁/R₂ consensus using FLASH. This inital process is not required when processing long read sequencing data from PacBio or Oxford Nanopore Technologies platforms. Instead, a target site reference is built, which describes the sequences for all expected on-target locations. Optionally, a target is built that contains an expected outcome of a homology directed repair (HDR) event. Next, the merged sequence reads are aligned to the target reference sequences using minimap2, (which was originally developed for rapid alignment of long reads (e.g., those generated by the Oxford Nanopore Technologies MinION). Reads aligning to each target are then re-aligned using a modified form of the Needleman-Wunsch aligner, called psnw. The modified aligner allows for improved detection of insertions and deletions resulting from DSB repair. All observed variants within a pre-defined distance of the DSB location are characterized and quantified. Finally, the results are summarized in tables and graphs. The various described programs, tools, and file types are familiar to and readily accessible to those having ordinary skill in the art. It should be understood that these programs, tools, and file types are exemplary and are not intended to be limiting. Other tools and file types could be used to practice the described processing and analysis.

In this analytical pipeline, the following improvements over prior methods are described. First, the use of minimap2 enables alignment of reads generated from both short and long read sequencers. Second, by constructing the expected outcome of the homology directed repair event, the ability to characterize perfect (i.e., correctly occurring) HDR events is improved. Third, use of the modified Needleman-Wunsch aligner that can accept a Cas-specific bonus matrix enables significantly improved indel characterization and percent (%) editing quantification over prior methods. Fourth, graphical visualization of the introduced allelic variants is improved. Fifth, a predicted repair event, as described in a prior tool, is compared against the observed repair, and the molecular pathways involved in the repair can be described.

In one embodiment, the processes described herein have the following advantageous uses:
Accurate characterization of indel profiles resulting from DSBs.
The fraction of reads containing an indel after a DSB is repaired is used to calculate the percentage of editing. This metric (% editing) is used to determine the effectiveness of a gRNA for use in CRISPR-Cas gene editing.
Accurate characterization of the resulting indel similarly improves the ability to identify the percentage of cellular chromosomes in a population of cells containing a frame-shifting mutation. Frame-shifting mutations modify proteins encoded by affected genes.
Accurate characterization of inserted sequences.
Accurate characterization of multiple mutations resulting from multiple gRNA/Cas9 (i.e., ribonucleoprotein complex) deliveries or dual-guide region modifications.
Analysis of indels sequenced on a long-read platform, such as MinION. Additionally, it allows phased characterization of both ends of large (>400 nt) insertion of deletion events, which occur after DSB repair.
Improved result visualization.

One embodiment described herein is a computer implemented process for identifying and characterizing double-stranded DNA break repair sites with improved accuracy, the process comprising executing on a processor the steps of: receiving sample sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics.

In one embodiment, edited genomic DNA is extracted and amplified using targeted multiplex PCR to enrich for the on- and predicted off-target loci. Amplicons are sequenced on an Illumina MiSeq. When using when processing paired-end reads from short read sequencing such as the illumina platform, the read pairs are merged into a single fragment (FLASH), mapped to the genome (minimap2), and binned by their alignment to expected amplicon positions. This step is not required for output from long read sequence data from PacBio or Oxford Nanopore Technologies platforms. Reads in each bin are re-aligned to the expected amplicon sequence after finding the cut site and creating a position specific gap open/extension bonus matrix to preferentially align indels closer to the cut site/expected indel profiles for each enzyme (CRISPAltRations code+psnw). Indels that intersected with a window upstream or downstream of the cut site were annotated. Percent editing is the sum of reads containing indels/total observed.

In some embodiments, the process described herein uses minimap2, which enables alignment of reads generated from both short and long read sequencers. Prior tools typically only accept short read sequencing data, such as those that are generated by Illumina sequencers. Others have used long read sequencing data to examine large insertions or deletions, but no stand-alone publicly available tools are believed to exist. Long read data handling is partially enabled by use of the minimap2 aligner. For example, the alignment results can be visualized, which shows identification of a blunt molecular insertion in DNA after a DSB repair.

In another embodiment, by constructing the expected outcome of the HDR event, the ability to characterize perfect HDR events is improved. A reference file, in FASTA format, contains each expected sequence target and modified sequence targets as well. The first step toward constructing this file involves creating a reference sequence index that enables reads to be aligned to each expected structural variant. For example, if one interrogates a region targeted for a DSB and double stranded DNA donor oligo to enable HDR, there are multiple different likely biological repair outcomes: perfect repair, HDR-mediated repair, NHEJ repair, and NHEJ repair with duplicate insertion. Other outcomes, such as template fragment or triple template insertions, are also possible. A similar reference file construction approach has been used by other tools, such as UDiTaS™.

In another embodiment, for short read sequences, a modified version of the Needleman-Wunsch algorithm is used to re-align reads against their expected target. The method described herein increases accuracy of alignments containing an indel (as annotated in alignment's CIGAR string). It significantly improves indel characterization and % editing quantification over prior methods. DNA sequence aligners such as minimap2 and Needleman-Wunsch approaches weigh indel alignments using fixed penalties for opening and extending gaps. This method is improved upon by re-aligning reads to their targets using position-specific gap open and extension penalties (enabled in a tool called “psnw”) such that alignments with indels favor positioning them overlapping or near the predicted DSB. This position specific matrix is set to reflect the actual characterized indel profile of the specific Cas enzyme being used for editing. Thus, indel base alignments are most highly favored at or near the predicted target cut site (variable scoring strategy). This method enables accurate realignment of indels, particularly those that occur in repetitive regions in the reference sequence. This approach improves the ability to identify the most biologically likely result.

In another embodiment, the processes described herein collect indels nearby the nuclease cut site and tag indels that intersect the cut site, or within a fixed distance. Some published accounts suggest a 1-2 nt fixed distance, but the data supporting those choices has been limited. In developing the embodiments described herein, the optimal distance (i.e., window size) around the cut site was studied using a set of Cas9-RNP treated and paired untreated control samples. It was observed that a 4-nt window for Cas9 or a 7-nt window for Cas12a provided the greatest sensitivity and provided an acceptable specificity. The larger window requirement for Cas12a is likely due to the mechanism of action; Cas12a implements a double strand break by producing two single strand breaks 5-bp away (leaving “sticky” ends). Thus, the process described herein can be expanded to other nucleases (e.g., CasX) having biological data to inform the target window size and enzymatic mechanism of action.

In another embodiment, graphical visualization of the allelic variation is improved. Downstream of the alignment step, several other analyses are performed that are unique to the described method. To generate an improved visualization, reads are deduplicated based on the identity of identified indel sequences within the CRISPR editing window post-alignment. Deduplicated reads are written back to a BAM file, and the frequency of each deduplicated read within the original population of reads is written to an associated BAM tag. After the file is indexed, indels in deduplicated reads and their associated frequencies can be visualized using the commonly available IGV tool.

The utility of the system and methods described herein is demonstrated by generating a synthetic set of 603 gRNA:amplicon pairs. At each target, 4000 read pairs (2×150 bp) are synthetically generated with a simulated Illumina MiSeq v3 platform error profile. In half of the reads, random indels are introduced based on a model generated off the observed editing profile for Cas9 and Cas12a. The synthetic data is analyzed using the CRISPAltRations system described herein. By implementing the method described herein, the ability to correctly characterize indels is improved by ˜15-20%. The algorithm described herein has increased accuracy because it produces a biologically informed selection of the best alignment in targets where multiple equally scored alignments are possible. Additionally, the method described herein more accurately calculates the percentage of modified DNA molecules. The process and strategy described herein is an important enhancement toward characterizing and quantifying indels introduced after DSB repair.

Another embodiment described herein is a computer implemented process for aligning biological sequences, the process comprising executing on a processor the steps of: receiving sample sequence data comprising a plurality of sequences; aligning the sequence data to a predicted target sequence using a matrix based on an enzyme specific position-specific scoring of a specific nuclease target site; outputting the alignment results as tables or graphics. In one aspect, the sequence data comprises sequences from a population of cells or subjects. In another aspect, the specific nuclease target sequence comprises a target site for one or more of Cas9, Cas12a, or other Cas enzymes. In another aspect, the matrix uses position-specific gap open and extension penalties.

Another embodiment described herein is a method for identifying and characterizing double-stranded DNA break repair sites with improved accuracy, the process comprising: extracting genomic DNA from a population of cells or tissue from a subject; amplifying the genomic DNA using multiplex PCR to produce amplicons enriched for target-site sequences; sequencing the amplicons and obtaining sample sequence data; subsequently executing on a processor, the steps of: receiving sample sequence data comprising a plurality of sequences; analyzing and merging of the sample sequence data and outputting merged sequences; developing target-site sequences containing predicted outcomes of repair events when a single-stranded or a double-stranded DNA oligonucleotide donor is provided and outputting the target predicted outcomes; binning the merged sequences with the target-site sequences or the optional target predicted outcomes using a mapper and outputting target-read alignments; re-aligning the binned target-read alignments to the target-site using an enzyme specific position-specific scoring matrix derived from biological data that is applied based on the position of a guide sequence and a canonical enzyme-specific cut site and producing a final alignment; analyzing the final alignment and identifying and quantifying mutations within a pre-defined sequence distance window from the canonical enzyme-specific cut sites; outputting the final alignment, analysis, and quantification results data as tables or graphics.

Many different arrangements of the various components and processes described herein as well as components or processes not shown, are possible without departing from the spirit and scope of the present disclosure. It should be understood that embodiments or aspects may include and otherwise be implemented by a combination of various hardware, software, or electronic components. For example, various microprocessors and application specific integrated circuits (“ASICs”) can be utilized, as can software of a variety of languages Also, servers and various computing devices can be used and can include one or more processing units, one or more computer-readable mediums, one or more input/output interfaces, and various connections (e.g., a system bus) connecting the components.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

### Synthetic Input Mix Generation

To investigate the utility of UMI incorporation with long read sequencing platforms, synthetic WT and HDR amplicons representing a sequence after a hypothetic perfect HDR insertion were generated from gene templates by PCR and mixed at known ratios. This allowed for a known HDR frequency in the synthetic DNA input before UMI sample prep to monitor PCR bias and correction via UMI consensus construction. To generate input DNA mixes for UMI amplification, the synthetic amplicons were PCR amplified with a high-fidelity polymerase (Platinum SuperFi II, Thermo Fisher) and limited cycle number (25 amplification cycles) to reduce the probability of polymerase error in the input DNA mixes. Synthetic templates representing three target sites within human genes were tested: HBB, TRAC, and SERPINC1. Again, with synthetic templates generated to represent knock-ins, the first two targets had 717 and 729 bp GFP insertions in the HDR amplicon, and SERPINC1 was tested with two HDR insertion lengths −500 bp and 1971 bp (Table 1). The ratios of WT:HDR amplicons in each input mix were quantified by Fragment Analyzer, qPCR, and sequenced using native barcoding (PCR-free) using an Oxford Nanopore Technologies MinION sequencer and analyzed using the CRISPAltRations pipeline to quantify percent HDR (data not shown). This represents the “expected” HDR in each sample prior to library preparation with UMIs incorporated.

TABLE 1
Synthetic Amplicons Used as Input Templates

| | | | WT | | | | | | HDR | |
|---|---|---|---|---|---|---|---|---|---|---|
| | Guide | SEQ | Amplicon | | SEQ | | Insertion | SEQ | Amplicon | SEQ |
| | Sequence | ID | Length | GC | ID | HDR | Length | ID | Length | ID |
| Target | Length | NO. | (bp) | (%) | NO. | Event | (bp) | NO. | (bp) | NO. |
| TRAC | 20 | 1 | 2027 | 48 | 2 | 729 bp | 729 | 3 | 2756 | 4 |
| | | | | | | insertion | | | | |
| HBB | 20 | 5 | 2715 | 40 | 6 | 717 bp | 717 | 7 | 3432 | 8 |
| | | | | | | insertion | | | | |
| SERPINC1 | 20 | 9 | 1991 | 51 | 10 | 500 bp | 500 | 11 | 2491 | 12 |
| | | | | | | insertion | | | | |
| SERPINC1 | 20 | 9 | 1991 | 51 | 10 | 2 kb | 1971 | 13 | 3962 | 14 |
| | | | | | | insertion | | | | |

### Sequences

| | |
|---|---|
| TRAC | |
| TRAC Guide | GAGAATCAAAATCGGTGAAT |
| sequence | |
| (SEQ ID NO: 1) | |
| TRAC WT | GCAGGAGGTCGGAAAGAATAAACAATGAGAGTCACATTAAAAACACAAAATCCTACGGAAATACTGAA |
| amplicon | GAATGAGTCTCAGCACTAAGGAAAAGCCTCCAGCAGCTCCTGCTTTCTGAGGGTGAAGGATAGACGCT |
| sequence | GTGGCTCTGCATGACTCACTAGCACTCTATCACGGCCATATTCTGGCAGGGTCAGTGGCTCCAACTAA |
| (SEQ ID NO: 2) | CATTTGTTTGGTACTTTACAGTTTATTAAATAGATGTTTATATGGAGAAGCTCTCATTTCTTTCTCAG |
| | AAGAGCCTGGCTAGGAAGGTGGATGAGGCACCATATTCATTTTGCAGGTGAAATTCCTGAGATGTAAG |
| | GAGCTGCTGTGACTTGCTCAAGGCCTTATATCGAGTAAACGGTAGTGCTGGGGCTTAGACGCAGGTGT |
| | TCTGATTTATAGTTCAAAACCTCTATCAATGAGAGAGCAATCTCCTGGTAATGTGATAGATTTCCCAA |
| | CTTAATGCCAACATACCATAAACCTCCCATTCTGCTAATGCCCAGCCTAAGTTGGGGAGACCACTCCA |
| | GATTCCAAGATGTACAGTTTGCTTTGCTGGGCCTTTTTCCCATGCCTGCCTTTACTCTGCCAGAGTTA |
| | TATTGCTGGGGTTTTGAAGAAGATCCTATTAAATAAAAGAATAAGCAGTATTATTAAGTAGCCCTGCA |
| | TTTCAGGTTTCCTTGAGTGGCAGGCCAGGCCTGGCCGTGAACGTTCACTGAAATCATGGCCTCTTGGC |
| | CAAGATTGATAGCTTGTGCCTGTCCCTGAGTCCCAGTCCATCACGAGCAGCTGGTTTCTAAGATGCTA |
| | TTTCCCGTATAAAGCATGAGACCGTGACTTGCCAGCCCCACAGAGCCCCGCCCTTGTCCATCACTGGC |
| | ATCTGGACTCCAGCCTGGGTTGGGGCAAAGAGGGAAATGAGATCATGTCCTAACCCTGATCCTCTTGT |
| | CCCACAGATATCCAGAACCCTGACCCTGCCGTGTACCAGCTGAGAGACTCTAAATCCAGTGACAAGTC |
| | TGTCTGCCTATTCACCGATTTTGATTCTCAAACAAATGTGTCACAAAGTAAGGATTCTGATGTGTATA |
| | TCACAGACAAAACTGTGCTAGACATGAGGTCTATGGACTTCAAGAGCAACAGTGCTGTGGCCTGGAGC |
| | AACAAATCTGACTTTGCATGTGCAAACGCCTTCAACAACAGCATTATTCCAGAAGACACCTTCTTCCC |
| | CAGCCCAGGTAAGGGCAGCTTTGGTGCCTTCGCAGGCTGTTTCCTTGCTTCAGGAATGGCCAGGTTCT |
| | GCCCAGAGCTCTGGTCAATGATGTCTAAAACTCCTCTGATTGGTGGTCTCGGCCTTATCCATTGCCAC |
| | CAAAACCCTCTTTTTACTAAGAAACAGTGAGCCTTGTTCTGGCAGTCCAGAGAATGACACGGGAAAAA |
| | AGCAGATGAAGAGAAGGTGGCAGGAGAGGGCACGTGGCCCAGCCTCAGTCTCTCCAACTGAGTTCCTG |
| | CCTGCCTGCCTTTGCTCAGACTGTTTGCCCCTTACTGCTCTTCTAGGCCTCATTCTAAGCCCCTTCTC |
| | CAAGTTGCCTCTCCTTATTTCTCCCTGTCTGCCAAAAAATCTTTCCCAGCTCACTAAGTCAGTCTCAC |
| | GCAGTCACTCATTAACCCACCAATCACTGATTGTGCCGGCACATGAATGCACCAGGTGTTGAAGTGGA |
| | GGAATTAAAAAGTCAGATGAGGGGTGTGCCCAGAGGAAGCACCATTCTAGTTGGGGGAGCCCATCTGT |
| | CAGCTGGGAAAAGTCCAAATAACTTCAGATTGGAATGTGTTTTAACTCAGGGTTGAGAAAACAGCTAC |
| | CTTCAGGACAAAAGTCAGGGAAGGGCTCTCTGAAGAAATGCTACTTGAAGATACCAGCCCTACCAAGG |
| | GCAGGGAGAGGACCCTATAGAGGCCTGGGACAGGAGCTCAATGAGAAAGGAGAAGAGCAGCAGGCATG |
| | AGTTGAATGAAGGAGGCAGGGCCGGGTCACAGGGCCTTCTAGGCCATGAGAGGGT |
| TRAC HDR | GGATCGGGTGGGACTAGTGGCAGCAAGGGCGAGGAGCTGTTCACCGGGGTGGTGCCCATCCTGGTCGA |
| insert sequence | GCTGGACGGCGACGTAAACGGCCACAAGTTCAGCGTGCGCGGCGAGGGCGAGGGCGATGCCACCAACG |
| (SEQ ID NO: 3) | GCAAGCTGACCCTGAAGTTCATCTGCACCACCGGCAAGCTGCCCGTGCCCTGGCCCACCCTCGTGACC |
| | ACCCTGACCTACGGCGTGCAGTGCTTCAGCCGCTACCCCGACCACATGAAGCGCCACGACTTCTTCAA |
| | GTCCGCCATGCCCGAAGGCTACGTCCAGGAGCGCACCATCAGCTTCAAGGACGACGGCACCTACAAGA |
| | CCCGCGCCGAGGTGAAGTTCGAGGGCGACACCCTGGTGAACCGCATCGAGCTGAAGGGCATCGACTTC |
| | AAGGAGGACGGCAACATCCTGGGGCACAAGCTGGAGTACAACTTCAACAGCCACAACGTCTATATCAC |
| | CGCCGACAAGCAGAAGAACGGCATCAAGGCCAACTTCAAGATCCGCCACAACGTGGAGGACGGCAGCG |
| | TGCAGCTCGCCGACCACTACCAGCAGAACACCCCCATCGGCGACGGCCCCGTGCTGCTGCCCGACAAC |
| | CACTACCTGAGCACCCAGTCCGTGCTGAGCAAAGACCCCAACGAGAAGCGCGATCACATGGTCCTGCT |
| | GGAGTTCGTGACCGCCGCCGGGATCACTGGAACCGGTGCTGGAAGTGGT |
| TRAC HDR | GCAGGAGGTCGGAAAGAATAAACAATGAGAGTCACATTAAAAACACAAAATCCTACGGAAATACTGAA |
| amplicon | GAATGAGTCTCAGCACTAAGGAAAAGCCTCCAGCAGCTCCTGCTTTCTGAGGGTGAAGGATAGACGCT |
| sequence | GTGGCTCTGCATGACTCACTAGCACTCTATCACGGCCATATTCTGGCAGGGTCAGTGGCTCCAACTAA |
| (SEQ ID NO: 4) | CATTTGTTTGGTACTTTACAGTTTATTAAATAGATGTTTATATGGAGAAGCTCTCATTTCTTTCTCAG |
| | AAGAGCCTGGCTAGGAAGGTGGATGAGGCACCATATTCATTTTGCAGGTGAAATTCCTGAGATGTAAG |
| | GAGCTGCTGTGACTTGCTCAAGGCCTTATATCGAGTAAACGGTAGTGCTGGGGCTTAGACGCAGGTGT |
| | TCTGATTTATAGTTCAAAACCTCTATCAATGAGAGAGCAATCTCCTGGTAATGTGATAGATTTCCCAA |
| | CTTAATGCCAACATACCATAAACCTCCCATTCTGCTAATGCCCAGCCTAAGTTGGGGAGACCACTCCA |
| | GATTCCAAGATGTACAGTTTGCTTTGCTGGGCCTTTTTCCCATGCCTGCCTTTACTCTGCCAGAGTTA |
| | TATTGCTGGGGTTTTGAAGAAGATCCTATTAAATAAAAGAATAAGCAGTATTATTAAGTAGCCCTGCA |
| | TTTCAGGTTTCCTTGAGTGGCAGGCCAGGCCTGGCCGTGAACGTTCACTGAAATCATGGCCTCTTGGC |
| | CAAGATTGATAGCTTGTGCCTGTCCCTGAGTCCCAGTCCATCACGAGCAGCTGGTTTCTAAGATGCTA |
| | TTTCCCGTATAAAGCATGAGACCGTGACTTGCCAGCCCCACAGAGCCCCGCCCTTGTCCATCACTGGC |
| | ATCTGGACTCCAGCCTGGGTTGGGGCAAAGAGGGAAATGAGATCATGTCCTAACCCTGATCCTCTTGT |
| | CCCACAGATATCCAGAACCCTGACCCTGCCGTGTACCAGCTGAGAGACTCTAAATCCAGTGACAAGTC |
| | TGTCTGCCTATTCACCGGGATCGGGTGGGACTAGTGGCAGCAAGGGCGAGGAGCTGTTCACCGGGGTG |
| | GTGCCCATCCTGGTCGAGCTGGACGGCGACGTAAACGGCCACAAGTTCAGCGTGCGCGGCGAGGGCGA |
| | GGGCGATGCCACCAACGGCAAGCTGACCCTGAAGTTCATCTGCACCACCGGCAAGCTGCCCGTGCCCT |
| | GGCCCACCCTCGTGACCACCCTGACCTACGGCGTGCAGTGCTTCAGCCGCTACCCCGACCACATGAAG |
| | CGCCACGACTTCTTCAAGTCCGCCATGCCCGAAGGCTACGTCCAGGAGCGCACCATCAGCTTCAAGGA |
| | CGACGGCACCTACAAGACCCGCGCCGAGGTGAAGTTCGAGGGCGACACCCTGGTGAACCGCATCGAGC |
| | TGAAGGGCATCGACTTCAAGGAGGACGGCAACATCCTGGGGCACAAGCTGGAGTACAACTTCAACAGC |
| | CACAACGTCTATATCACCGCCGACAAGCAGAAGAACGGCATCAAGGCCAACTTCAAGATCCGCCACAA |
| | CGTGGAGGACGGCAGCGTGCAGCTCGCCGACCACTACCAGCAGAACACCCCCATCGGCGACGGCCCCG |
| | TGCTGCTGCCCGACAACCACTACCTGAGCACCCAGTCCGTGCTGAGCAAAGACCCCAACGAGAAGCGC |
| | GATCACATGGTCCTGCTGGAGTTCGTGACCGCCGCCGGGATCACTGGAACCGGTGCTGGAAGTGGTAT |
| | TTTGATTCTCAAACAAATGTGTCACAAAGTAAGGATTCTGATGTGTATATCACAGACAAAACTGTGCT |
| | AGACATGAGGTCTATGGACTTCAAGAGCAACAGTGCTGTGGCCTGGAGCAACAAATCTGACTTTGCAT |
| | GTGCAAACGCCTTCAACAACAGCATTATTCCAGAAGACACCTTCTTCCCCAGCCCAGGTAAGGGCAGC |
| | TTTGGTGCCTTCGCAGGCTGTTTCCTTGCTTCAGGAATGGCCAGGTTCTGCCCAGAGCTCTGGTCAAT |
| | GATGTCTAAAACTCCTCTGATTGGTGGTCTCGGCCTTATCCATTGCCACCAAAACCCTCTTTTTACTA |
| | AGAAACAGTGAGCCTTGTTCTGGCAGTCCAGAGAATGACACGGGAAAAAAGCAGATGAAGAGAAGGTG |
| | GCAGGAGAGGGCACGTGGCCCAGCCTCAGTCTCTCCAACTGAGTTCCTGCCTGCCTGCCTTTGCTCAG |
| | ACTGTTTGCCCCTTACTGCTCTTCTAGGCCTCATTCTAAGCCCCTTCTCCAAGTTGCCTCTCCTTATT |
| | TCTCCCTGTCTGCCAAAAAATCTTTCCCAGCTCACTAAGTCAGTCTCACGCAGTCACTCATTAACCCA |
| | CCAATCACTGATTGTGCCGGCACATGAATGCACCAGGTGTTGAAGTGGAGGAATTAAAAAGTCAGATG |
| | AGGGGTGTGCCCAGAGGAAGCACCATTCTAGTTGGGGGAGCCCATCTGTCAGCTGGGAAAAGTCCAAA |
| | TAACTTCAGATTGGAATGTGTTTTAACTCAGGGTTGAGAAAACAGCTACCTTCAGGACAAAAGTCAGG |
| | GAAGGGCTCTCTGAAGAAATGCTACTTGAAGATACCAGCCCTACCAAGGGCAGGGAGAGGACCCTATA |
| | GAGGCCTGGGACAGGAGCTCAATGAGAAAGGAGAAGAGCAGCAGGCATGAGTTGAATGAAGGAGGCAG |
| | GGCCGGGTCACAGGGCCTTCTAGGCCATGAGAGGGT |
| HBB | |
| HBB Guide | CTTGCCCCACAGGGCAGTAA |
| sequence | |
| (SEQ ID NO: 5) | |
| HBB WT | AGCAGGAAGCAGAACTCTGCACTTCAAAAGTTTTTCCTCACCTGAGGAGTTAATTTAGTACAAGGGGA |
| amplicon | AAAAGTACAGGGGGATGGGAGAAAGGCGATCACGTTGGGAAGCTATAGAGAAAGAAGAGTAAATTTTA |
| sequence | GTAAAGGAGGTTTAAACAAACAAAATATAAAGAGAAATAGGAACTTGAATCAAGGAAATGATTTTAAA |
| (SEQ ID NO: 6) | ACGCAGTATTCTTAGTGGACTAGAGGAAAAAAATAATCTGAGCCAAGTAGAAGACCTTTTCCCCTCCT |
| | ACCCCTACTTTCTAAGTCACAGAGGCTTTTTGTTCCCCCAGACACTCTTGCAGATTAGTCCAGGCAGA |
| | AACAGTTAGATGTCCCCAGTTAACCTCCTATTTGACACCACTGATTACCCCATTGATAGTCACACTTT |
| | GGGTTGTAAGTGACTTTTTATTTATTTGTATTTTTGACTGCATTAAGAGGTCTCTAGTTTTTTATCTC |
| | TTGTTTCCCAAAACCTAATAAGTAACTAATGCACAGAGCACATTGATTTGTATTTATTCTATTTTTAG |
| | ACATAATTTATTAGCATGCATGAGCAAATTAAGAAGAACAACAACAAATGAATGCATATATATGTATA |
| | TGTACGTGTGTATATATACACACATATGTATATATATTTTTTCTTTTCTTACCAGAAGGTTTTAATCC |
| | AAATAAGGAGAAGATATGCTTAGAACCGAGGTAGAGTTTTCATCCATTCTGTCCTGTAAGTATTTTGC |
| | ATATTCTGGAGACGCAGGAAGAGATCCATCTACATATCCCAAAGCTGAATTATGGTAGACAAAACTCT |
| | TCCACTTTTAGTGCATCAACTTCTTATTTGTGTAATAAGAAAATTGGGAAAACGATCTTCAATATGCT |
| | TACCAAGCTGTGATTCCAAATATTACGTAAATACACTTGCAAAGGAGGATGTTTTTAGTAGCAATTTG |
| | TACTGATGGTATGGGGCCAAGAGATATATCTTAGAGGGAGGGCTGAGGGTTTGAAGTCCAACTCCTAA |
| | GCCAGTGCCAGAAGAGCCAAGGACAGGTACGGCTGTCATCACTTAGACCTCACCCTGTGGAGCCACAC |
| | CCTAGGGTTGGCCAATCTACTCCCAGGAGCAGGGAGGGCAGGAGCCAGGGCTGGGCATAAAAGTCAGG |
| | GCAGAGCCATCTATTGCTTACATTTGCTTCTGACACAACTGTGTTCACTAGCAACCTCAAACAGACAC |
| | CATGGTGCATCTGACTCCTGAGGAGAAGTCTGCCGTTACTGCCCTGTGGGGCAAGGTGAACGTGGATG |
| | AAGTTGGTGGTGAGGCCCTGGGCAGGTTGGTATCAAGGTTACAAGACAGGTTTAAGGAGACCAATAGA |
| | AACTGGGCATGTGGAGACAGAGAAGACTCTTGGGTTTCTGATAGGCACTGACTCTCTCTGCCTATTGG |
| | TCTATTTTCCCACCCTTAGGCTGCTGGTGGTCTACCCTTGGACCCAGAGGTTCTTTGAGTCCTTTGGG |
| | GATCTGTCCACTCCTGATGCTGTTATGGGCAACCCTAAGGTGAAGGCTCATGGCAAGAAAGTGCTCGG |
| | TGCCTTTAGTGATGGCCTGGCTCACCTGGACAACCTCAAGGGCACCTTTGCCACACTGAGTGAGCTGC |
| | ACTGTGACAAGCTGCACGTGGATCCTGAGAACTTCAGGGTGAGTCTATGGGACGCTTGATGTTTTCTT |
| | TCCCCTTCTTTTCTATGGTTAAGTTCATGTCATAGGAAGGGGATAAGTAACAGGGTACAGTTTAGAAT |
| | GGGAAACAGACGAATGATTGCATCAGTGTGGAAGTCTCAGGATCGTTTTAGTTTCTTTTATTTGCTGT |
| | TCATAACAATTGTTTTCTTTTGTTTAATTCTTGCTTTCTTTTTTTTTCTTCTCCGCAATTTTTACTAT |
| | TATACTTAATGCCTTAACATTGTGTATAACAAAAGGAAATATCTCTGAGATACATTAAGTAACTTAAA |
| | AAAAAACTTTACACAGTCTGCCTAGTACATTACTATTTGGAATATATGTGTGCTTATTTGCATATTCA |
| | TAATCTCCCTACTTTATTTTCTTTTATTTTTAATTGATACATAATCATTATACATATTTATGGGTTAA |
| | AGTGTAATGTTTTAATATGTGTACACATATTGACCAAATCAGGGTAATTTTGCATTTGTAATTTTAAA |
| | AAATGCTTTCTTCTTTTAATATACTTTTTTGTTTATCTTATTTCTAATACTTTCCCTAATCTCTTTCT |
| | TTCAGGGCAATAATGATACAATGTATCATGCCTCTTTGCACCATTCTAAAGAATAACAGTGATAATTT |
| | CTGGGTTAAGGCAATAGCAATATCTCTGCATATAAATATTTCTGCATATAAATTGTAACTGATGTAAG |
| | AGGTTTCATATTGCTAATAGCAGCTACAATCCAGCTACCATTCTGCTTTTATTTTATGGTTGGGATAA |
| | GGCTGGATTATTCTGAGTCCAAGCTAGGCCCTTTTGCTAATCATGTTCATACCTCTTATCTTCCTCCC |
| | ACAGCTCCTGGGCAACGTGCTGGTCTGTGTGCTGGCCCATCACTTTGGCAAAGAATTCACCCCACCAG |
| | TGCAGGCTGCATATCAGAAAGTGGTGGCTGGTGTGGCTAATGCCCTGGCCCACAAGTATCACTAAGCT |
| | CGCTTTCTTGCTGTCCAATTTCTATTAAAGGTTCCTTTGTTCCCTAAGTCCAACTACTAAACT |
| HBB HDR insert | ATGGTGAGCAAGGGCGAGGAGCTGTTCACCGGGGTGGTGCCCATCCTGGTCGAGCTGGACGGCGACGT |
| sequence | AAACGGCCACAAGTTCAGCGTGTCCGGCGAGGGCGAGGGCGATGCCACCTACGGCAAGCTGACCCTGA |
| (SEQ ID NO: 7) | AGTTCATCTGCACCACCGGCAAGCTGCCCGTGCCCTGGCCCACCCTCGTGACCACCCTGACCTACGGC |
| | GTGCAGTGCTTCAGCCGCTACCCCGACCACATGAAGCAGCACGACTTCTTCAAGTCCGCCATGCCCGA |
| | AGGCTACGTCCAGGAGCGCACCATCTTCTTCAAGGACGACGGCAACTACAAGACCCGCGCCGAGGTGA |
| | AGTTCGAGGGCGACACCCTGGTGAACCGCATCGAGCTGAAGGGCATCGACTTCAAGGAGGACGGCAAC |
| | ATCCTGGGGCACAAGCTGGAGTACAACTACAACAGCCACAACGTCTATATCATGGCCGACAAGCAGAA |
| | GAACGGCATCAAGGTGAACTTCAAGATCCGCCACAACATCGAGGACGGCAGCGTGCAGCTCGCCGACC |
| | ACTACCAGCAGAACACCCCCATCGGCGACGGCCCCGTGCTGCTGCCCGACAACCACTACCTGAGCACC |
| | CAGTCCGCCCTGAGCAAAGACCCCAACGAGAAGCGCGATCACATGGTCCTGCTGGAGTTCGTGACCGC |
| | CGCCGGGATCACTCTCGGCATGGACGAGCTGTACAAG |
| HBB HDR | AGCAGGAAGCAGAACTCTGCACTTCAAAAGTTTTTCCTCACCTGAGGAGTTAATTTAGTACAAGGGGA |
| amplicon | AAAAGTACAGGGGGATGGGAGAAAGGCGATCACGTTGGGAAGCTATAGAGAAAGAAGAGTAAATTTTA |
| sequence | GTAAAGGAGGTTTAAACAAACAAAATATAAAGAGAAATAGGAACTTGAATCAAGGAAATGATTTTAAA |
| (SEQ ID NO: 8) | ACGCAGTATTCTTAGTGGACTAGAGGAAAAAAATAATCTGAGCCAAGTAGAAGACCTTTTCCCCTCCT |
| | ACCCCTACTTTCTAAGTCACAGAGGCTTTTTGTTCCCCCAGACACTCTTGCAGATTAGTCCAGGCAGA |
| | AACAGTTAGATGTCCCCAGTTAACCTCCTATTTGACACCACTGATTACCCCATTGATAGTCACACTTT |
| | GGGTTGTAAGTGACTTTTTATTTATTTGTATTTTTGACTGCATTAAGAGGTCTCTAGTTTTTTATCTC |
| | TTGTTTCCCAAAACCTAATAAGTAACTAATGCACAGAGCACATTGATTTGTATTTATTCTATTTTTAG |
| | ACATAATTTATTAGCATGCATGAGCAAATTAAGAAGAACAACAACAAATGAATGCATATATATGTATA |
| | TGTACGTGTGTATATATACACACATATGTATATATATTTTTTCTTTTCTTACCAGAAGGTTTTAATCC |
| | AAATAAGGAGAAGATATGCTTAGAACCGAGGTAGAGTTTTCATCCATTCTGTCCTGTAAGTATTTTGC |
| | ATATTCTGGAGACGCAGGAAGAGATCCATCTACATATCCCAAAGCTGAATTATGGTAGACAAAACTCT |
| | TCCACTTTTAGTGCATCAACTTCTTATTTGTGTAATAAGAAAATTGGGAAAACGATCTTCAATATGCT |
| | TACCAAGCTGTGATTCCAAATATTACGTAAATACACTTGCAAAGGAGGATGTTTTTAGTAGCAATTTG |
| | TACTGATGGTATGGGGCCAAGAGATATATCTTAGAGGGAGGGCTGAGGGTTTGAAGTCCAACTCCTAA |
| | GCCAGTGCCAGAAGAGCCAAGGACAGGTACGGCTGTCATCACTTAGACCTCACCCTGTGGAGCCACAC |
| | CCTAGGGTTGGCCAATCTACTCCCAGGAGCAGGGAGGGCAGGAGCCAGGGCTGGGCATAAAAGTCAGG |
| | GCAGAGCCATCTATTGCTTACATTTGCTTCTGACACAACTGTGTTCACTAGCAACCTCAAACAGACAC |
| | CATGGTGCATCTGACTCCTGAGGAGAAGTCTGCCGTTAATGGTGAGCAAGGGCGAGGAGCTGTTCACC |
| | GGGGTGGTGCCCATCCTGGTCGAGCTGGACGGCGACGTAAACGGCCACAAGTTCAGCGTGTCCGGCGA |
| | GGGCGAGGGCGATGCCACCTACGGCAAGCTGACCCTGAAGTTCATCTGCACCACCGGCAAGCTGCCCG |
| | TGCCCTGGCCCACCCTCGTGACCACCCTGACCTACGGCGTGCAGTGCTTCAGCCGCTACCCCGACCAC |
| | ATGAAGCAGCACGACTTCTTCAAGTCCGCCATGCCCGAAGGCTACGTCCAGGAGCGCACCATCTTCTT |
| | CAAGGACGACGGCAACTACAAGACCCGCGCCGAGGTGAAGTTCGAGGGCGACACCCTGGTGAACCGCA |
| | TCGAGCTGAAGGGCATCGACTTCAAGGAGGACGGCAACATCCTGGGGCACAAGCTGGAGTACAACTAC |
| | AACAGCCACAACGTCTATATCATGGCCGACAAGCAGAAGAACGGCATCAAGGTGAACTTCAAGATCCG |
| | CCACAACATCGAGGACGGCAGCGTGCAGCTCGCCGACCACTACCAGCAGAACACCCCCATCGGCGACG |
| | GCCCCGTGCTGCTGCCCGACAACCACTACCTGAGCACCCAGTCCGCCCTGAGCAAAGACCCCAACGAG |
| | AAGCGCGATCACATGGTCCTGCTGGAGTTCGTGACCGCCGCCGGGATCACTCTCGGCATGGACGAGCT |
| | GTACAAGCTGCCCTGTGGGGCAAGGTGAACGTGGATGAAGTTGGTGGTGAGGCCCTGGGCAGGTTGGT |
| | ATCAAGGTTACAAGACAGGTTTAAGGAGACCAATAGAAACTGGGCATGTGGAGACAGAGAAGACTCTT |
| | GGGTTTCTGATAGGCACTGACTCTCTCTGCCTATTGGTCTATTTTCCCACCCTTAGGCTGCTGGTGGT |
| | CTACCCTTGGACCCAGAGGTTCTTTGAGTCCTTTGGGGATCTGTCCACTCCTGATGCTGTTATGGGCA |
| | ACCCTAAGGTGAAGGCTCATGGCAAGAAAGTGCTCGGTGCCTTTAGTGATGGCCTGGCTCACCTGGAC |
| | AACCTCAAGGGCACCTTTGCCACACTGAGTGAGCTGCACTGTGACAAGCTGCACGTGGATCCTGAGAA |
| | CTTCAGGGTGAGTCTATGGGACGCTTGATGTTTTCTTTCCCCTTCTTTTCTATGGTTAAGTTCATGTC |
| | ATAGGAAGGGGATAAGTAACAGGGTACAGTTTAGAATGGGAAACAGACGAATGATTGCATCAGTGTGG |
| | AAGTCTCAGGATCGTTTTAGTTTCTTTTATTTGCTGTTCATAACAATTGTTTTCTTTTGTTTAATTCT |
| | TGCTTTCTTTTTTTTTCTTCTCCGCAATTTTTACTATTATACTTAATGCCTTAACATTGTGTATAACA |
| | AAAGGAAATATCTCTGAGATACATTAAGTAACTTAAAAAAAAACTTTACACAGTCTGCCTAGTACATT |
| | ACTATTTGGAATATATGTGTGCTTATTTGCATATTCATAATCTCCCTACTTTATTTTCTTTTATTTTT |
| | AATTGATACATAATCATTATACATATTTATGGGTTAAAGTGTAATGTTTTAATATGTGTACACATATT |
| | GACCAAATCAGGGTAATTTTGCATTTGTAATTTTAAAAAATGCTTTCTTCTTTTAATATACTTTTTTG |
| | TTTATCTTATTTCTAATACTTTCCCTAATCTCTTTCTTTCAGGGCAATAATGATACAATGTATCATGC |
| | CTCTTTGCACCATTCTAAAGAATAACAGTGATAATTTCTGGGTTAAGGCAATAGCAATATCTCTGCAT |
| | ATAAATATTTCTGCATATAAATTGTAACTGATGTAAGAGGTTTCATATTGCTAATAGCAGCTACAATC |
| | CAGCTACCATTCTGCTTTTATTTTATGGTTGGGATAAGGCTGGATTATTCTGAGTCCAAGCTAGGCCC |
| | TTTTGCTAATCATGTTCATACCTCTTATCTTCCTCCCACAGCTCCTGGGCAACGTGCTGGTCTGTGTG |
| | CTGGCCCATCACTTTGGCAAAGAATTCACCCCACCAGTGCAGGCTGCATATCAGAAAGTGGTGGCTGG |
| | TGTGGCTAATGCCCTGGCCCACAAGTATCACTAAGCTCGCTTTCTTGCTGTCCAATTTCTATTAAAGG |
| | TTCCTTTGTTCCCTAAGTCCAACTACTAAACT |
| SERPINC1 | |
| SERPINC1 | ACCTCTGGAAAAAGGTAAGA |
| Guide sequence | |
| (SEQ ID NO: 9) | |
| SERPINC1 WT | CGCATTCTGTCTCCTGATCCCCCAGTAGAGTTTTGCTAAGTATTTCCCAGCTGCTCACACCCCTTAGA |
| amplicon | AACGCGCTTGGCATGCACCCCGAGGCCCTGCTCTTCTCTCCCTGTCCCACCACTTCAGGGCTGCTGGG |
| sequence | GAATGGGTCTCTCTGTGGGCCACAGGTGTAACCATTGTGTTTTCCTTGTCTGTGCCAGGGACACCTTG |
| (SEQ ID NO: 10) | GCACTCAGATGCCTGAAGGTAGCAGCTTGTCCCTCTTTGCCTTCTCTAATTAGATATTCCTCTCTCCA |
| | TAAAGAAAACTATGAGAGAGGGTGGGTATGAACCAAGTTTGTTTCCTTGGTTAGTTTCCTAACCAAGT |
| | TTGAGGGTATGAACATACTCTCCTTTTCCTTTTCTATAAAGCTGAGGAGAAGAGTGAGGGAGTGTGGG |
| | CAAGAGAGGTGGCTCAGGCTTTCCCTGGGCCTGATTGAACTTTAAAACTTCTCTACTAATTAAACAAC |
| | ACTGGGCTCTACACTTTGCTTAACCCTGGGAACTGGTCATCAGCCTTTGACCTCAGTTCCCCCTCCTG |
| | ACCAGCTCTCTGCCCCACCCTGTCCTCTGGAACCTCTGCGAGATTTAGAGGAAAGAACCAGTTTTCAG |
| | GCGGATTGCCTCAGATCACACTATCTCCACTTGCCCAGCCCTGTGGAAGATTAGCGGCCATGTATTCC |
| | AATGTGATAGGAACTGTAACCTCTGGAAAAAGGTAAGAGGGGTGAGCTTTCCCCTTGCCTGCCCCTAC |
| | TGGGTTTTGTGACCTCCAAAGGACTCACAGGAATGACCTCCAACACCTTTGAGAAGACCAGGCCCTCT |
| | CCCTGGTAGTTACAGTCAAAGACCTGTTTGGAAGACGTCATTTCAAGTGCTCTCCCTCCCACCCCACC |
| | TCTTGGGGTAAGGCCTTTCCTAAGCTACCCCTTGGGTCCCTAGCCTAAGAAACAAGGGGGATGTCATC |
| | CCTGGTGTAAAGATGCTGTGCAGGAAGTCAGCACTCACGGGATCCAGGGGACGCTCCAAGGGGAATCC |
| | CCAGGGCCTGCCATCCATCCGGGAAGAGAGCAAATGCTACCCATGAGGACCTCCTCACTCCCTTTTTG |
| | CTCTTTCTTCCACTCAGATCCACCCCACTCCACCCCCACCCAAATCCCAGTGACCTTTGACTAAAGGG |
| | CCAAAACTGCTTCCTTTTCTCACAATGAGAGTTGTCCCTCCCTCAATGCCACACACACTCCCTTCTTC |
| | ATCTGAGTTGTCACAGGAGGCTAGAAACGGGGTGGTGGCACAACTGTCTTGGTTTTAATTTGTGCTTC |
| | ATAGCCCTCCCAGGGTCCTCTCAGCCTCAAATTGCATTTCCAAATGTAGTTGAAGGACAGAGTGGGCA |
| | ACCGAAGGCAGTGGAGATGGGAAGATGAATGGCAGGGTCCTCTCCTCTCTCTCTCTGCTTCTTCAGCC |
| | TGCCTTCCACATCTCCCTTGGTGCCGCTGCTTCTCTCCGGCTTTGCACCTCTGTTCTTGAAAGGGCTG |
| | CAGAACTGGACTCAGACCACGCAAGAAGGCAAGTCCCCCTCAGCTGCCCCAGCTTCCAGCCAGCCCCA |
| | GGCTTGCCCAACGGACCACGTCCGTGAATCTGCACTGGGTGCCTGTCTTTCTCTCCCAGGAGAAGATG |
| | GGAAGATCCAGTACCCACACACAGACCCCCTTGTGTACACGCAGGAACCATAAACCAGCTGGAGGCAG |
| | CCCCTGCCCCACCCTGTCTTATCTACAAAAAATATTACAAGAGACTTTATCTCTTGATTTGCTTCATC |
| | GAGTGTCCCAACTACCTCATTTTTTTAAAATGTGAAATTAGCTTCATTTACCTTCATTGAATCCATGT |
| | TGGCGACTATTAAAAATTCCAGGCAATAAAAAGGGATGAGAGCCTGAACTAAAGCAGTGGCAATAACT |
| | GGTGAAAGAGTAAAAAAACAGATCTGATTGACTCTGGGGTGAACTGATTGACTCTCGCGTTTGACTAA |
| | ATGAGGAGGAGAGAGGGAG |
| SERPINC1 HDR | CGAATTCGAGGGCAGAGGCAGTCTGCTGACATGCGGTGACGTGGAAGAGAATCCCGGCCCTTCTAGAA |
| insert sequence | TGGTTAGCAAGGGCGAGGAGCTGTTCACCGGGGTGGTGCCCATCCTGGTCGAGCTGGACGGCGACGTA |
| (SEQ ID NO: 11) | AACGGCCACAAGTTCAGCGTGTCCGGCGAGGGAGAGGGCGATGCCACCTACGGCAAGCTGACCCTGAA |
| | GTTCATCTGCACCACCGGCAAGCTGCCAGTGCCCTGGCCTACCCTCGTGACCACCCTGACCTAACTGT |
| | GCCTTCTAGTTGCCAGCCATCTGTTGTTTGCGCCTCACTCGTGCCTTCATTGACCCTGGAAGGTGCCA |
| | CTCCCACTGTCCTTTCCTAATAAAATGAGGAAATTGCATCGCATTGTCTGAGTAGGTGTCATTCTATT |
| | CTGGCGTATCGAGTGGCTCAGGACAGCAAGAGCGAGGATTGGGAAGACAATAGCAGGCATGCTGGGGA |
| | TGCGGTGGGCTCTATGGCGGTACC |
| SERPINC1 HDR | CGCATTCTGTCTCCTGATCCCCCAGTAGAGTTTTGCTAAGTATTTCCCAGCTGCTCACACCCCTTAGA |
| amplicon | AACGCGCTTGGCATGCACCCCGAGGCCCTGCTCTTCTCTCCCTGTCCCACCACTTCAGGGCTGCTGGG |
| sequence | GAATGGGTCTCTCTGTGGGCCACAGGTGTAACCATTGTGTTTTCCTTGTCTGTGCCAGGGACACCTTG |
| (SEQ ID NO: 12) | GCACTCAGATGCCTGAAGGTAGCAGCTTGTCCCTCTTTGCCTTCTCTAATTAGATATTCCTCTCTCCA |
| | TAAAGAAAACTATGAGAGAGGGTGGGTATGAACCAAGTTTGTTTCCTTGGTTAGTTTCCTAACCAAGT |
| | TTGAGGGTATGAACATACTCTCCTTTTCCTTTTCTATAAAGCTGAGGAGAAGAGTGAGGGAGTGTGGG |
| | CAAGAGAGGTGGCTCAGGCTTTCCCTGGGCCTGATTGAACTTTAAAACTTCTCTACTAATTAAACAAC |
| | ACTGGGCTCTACACTTTGCTTAACCCTGGGAACTGGTCATCAGCCTTTGACCTCAGTTCCCCCTCCTG |
| | ACCAGCTCTCTGCCCCACCCTGTCCTCTGGAACCTCTGCGAGATTTAGAGGAAAGAACCAGTTTTCAG |
| | GCGGATTGCCTCAGATCACACTATCTCCACTTGCCCAGCCCTGTGGAAGATTAGCGGCCATGTATTCC |
| | AATGTGATAGGAACTGTAACCTCTGGAAAAAGGTAcGAATTCGAGGGCAGAGGCAGTCTGCTGACATG |
| | CGGTGACGTGGAAGAGAATCCCGGCCCTTCTAGAATGGTTAGCAAGGGCGAGGAGCTGTTCACCGGGG |
| | TGGTGCCCATCCTGGTCGAGCTGGACGGCGACGTAAACGGCCACAAGTTCAGCGTGTCCGGCGAGGGA |
| | GAGGGCGATGCCACCTACGGCAAGCTGACCCTGAAGTTCATCTGCACCACCGGCAAGCTGCCAGTGCC |
| | CTGGCCTACCCTCGTGACCACCCTGACCTAACTGTGCCTTCTAGTTGCCAGCCATCTGTTGTTTGCGC |
| | CTCACTCGTGCCTTCATTGACCCTGGAAGGTGCCACTCCCACTGTCCTTTCCTAATAAAATGAGGAAA |
| | TTGCATCGCATTGTCTGAGTAGGTGTCATTCTATTCTGGCGTATCGAGTGGCTCAGGACAGCAAGAGC |
| | GAGGATTGGGAAGACAATAGCAGGCATGCTGGGGATGCGGTGGGCTCTATGGCGGTACCAGAGGGGTG |
| | AGCTTTCCCCTTGCCTGCCCCTACTGGGTTTTGTGACCTCCAAAGGACTCACAGGAATGACCTCCAAC |
| | ACCTTTGAGAAGACCAGGCCCTCTCCCTGGTAGTTACAGTCAAAGACCTGTTTGGAAGACGTCATTTC |
| | AAGTGCTCTCCCTCCCACCCCACCTCTTGGGGTAAGGCCTTTCCTAAGCTACCCCTTGGGTCCCTAGC |
| | CTAAGAAACAAGGGGGATGTCATCCCTGGTGTAAAGATGCTGTGCAGGAAGTCAGCACTCACGGGATC |
| | CAGGGGACGCTCCAAGGGGAATCCCCAGGGCCTGCCATCCATCCGGGAAGAGAGCAAATGCTACCCAT |
| | GAGGACCTCCTCACTCCCTTTTTGCTCTTTCTTCCACTCAGATCCACCCCACTCCACCCCCACCCAAA |
| | TCCCAGTGACCTTTGACTAAAGGGCCAAAACTGCTTCCTTTTCTCACAATGAGAGTTGTCCCTCCCTC |
| | AATGCCACACACACTCCCTTCTTCATCTGAGTTGTCACAGGAGGCTAGAAACGGGGTGGTGGCACAAC |
| | TGTCTTGGTTTTAATTTGTGCTTCATAGCCCTCCCAGGGTCCTCTCAGCCTCAAATTGCATTTCCAAA |
| | TGTAGTTGAAGGACAGAGTGGGCAACCGAAGGCAGTGGAGATGGGAAGATGAATGGCAGGGTCCTCTC |
| | CTCTCTCTCTCTGCTTCTTCAGCCTGCCTTCCACATCTCCCTTGGTGCCGCTGCTTCTCTCCGGCTTT |
| | GCACCTCTGTTCTTGAAAGGGCTGCAGAACTGGACTCAGACCACGCAAGAAGGCAAGTCCCCCTCAGC |
| | TGCCCCAGCTTCCAGCCAGCCCCAGGCTTGCCCAACGGACCACGTCCGTGAATCTGCACTGGGTGCCT |
| | GTCTTTCTCTCCCAGGAGAAGATGGGAAGATCCAGTACCCACACACAGACCCCCTTGTGTACACGCAG |
| | GAACCATAAACCAGCTGGAGGCAGCCCCTGCCCCACCCTGTCTTATCTACAAAAAATATTACAAGAGA |
| | CTTTATCTCTTGATTTGCTTCATCGAGTGTCCCAACTACCTCATTTTTTTAAAATGTGAAATTAGCTT |
| | CATTTACCTTCATTGAATCCATGTTGGCGACTATTAAAAATTCCAGGCAATAAAAAGGGATGAGAGCC |
| | TGAACTAAAGCAGTGGCAATAACTGGTGAAAGAGTAAAAAAACAGATCTGATTGACTCTGGGGTGAAC |
| | TGATTGACTCTCGCGTTTGACTAAATGAGGAGGAGAGAGGGAG |
| SERPINC1 | |
| SERPINC1 | ACCTCTGGAAAAAGGTAAGA |
| Guide sequence | |
| (SEQ ID NO: 9) | |
| SERPINC1 WT | CGCATTCTGTCTCCTGATCCCCCAGTAGAGTTTTGCTAAGTATTTCCCAGCTGCTCACACCCCTTAGA |
| (SEQ ID NO: 10) | AACGCGCTTGGCATGCACCCCGAGGCCCTGCTCTTCTCTCCCTGTCCCACCACTTCAGGGCTGCTGGG |
| | GAATGGGTCTCTCTGTGGGCCACAGGTGTAACCATTGTGTTTTCCTTGTCTGTGCCAGGGACACCTTG |
| | GCACTCAGATGCCTGAAGGTAGCAGCTTGTCCCTCTTTGCCTTCTCTAATTAGATATTCCTCTCTCCA |
| | TAAAGAAAACTATGAGAGAGGGTGGGTATGAACCAAGTTTGTTTCCTTGGTTAGTTTCCTAACCAAGT |
| | TTGAGGGTATGAACATACTCTCCTTTTCCTTTTCTATAAAGCTGAGGAGAAGAGTGAGGGAGTGTGGG |
| | CAAGAGAGGTGGCTCAGGCTTTCCCTGGGCCTGATTGAACTTTAAAACTTCTCTACTAATTAAACAAC |
| | ACTGGGCTCTACACTTTGCTTAACCCTGGGAACTGGTCATCAGCCTTTGACCTCAGTTCCCCCTCCTG |
| | ACCAGCTCTCTGCCCCACCCTGTCCTCTGGAACCTCTGCGAGATTTAGAGGAAAGAACCAGTTTTCAG |
| | GCGGATTGCCTCAGATCACACTATCTCCACTTGCCCAGCCCTGTGGAAGATTAGCGGCCATGTATTCC |
| | AATGTGATAGGAACTGTAACCTCTGGAAAAAGGTAAGAGGGGTGAGCTTTCCCCTTGCCTGCCCCTAC |
| | TGGGTTTTGTGACCTCCAAAGGACTCACAGGAATGACCTCCAACACCTTTGAGAAGACCAGGCCCTCT |
| | CCCTGGTAGTTACAGTCAAAGACCTGTTTGGAAGACGTCATTTCAAGTGCTCTCCCTCCCACCCCACC |
| | TCTTGGGGTAAGGCCTTTCCTAAGCTACCCCTTGGGTCCCTAGCCTAAGAAACAAGGGGGATGTCATC |
| | CCTGGTGTAAAGATGCTGTGCAGGAAGTCAGCACTCACGGGATCCAGGGGACGCTCCAAGGGGAATCC |
| | CCAGGGCCTGCCATCCATCCGGGAAGAGAGCAAATGCTACCCATGAGGACCTCCTCACTCCCTTTTTG |
| | CTCTTTCTTCCACTCAGATCCACCCCACTCCACCCCCACCCAAATCCCAGTGACCTTTGACTAAAGGG |
| | CCAAAACTGCTTCCTTTTCTCACAATGAGAGTTGTCCCTCCCTCAATGCCACACACACTCCCTTCTTC |
| | ATCTGAGTTGTCACAGGAGGCTAGAAACGGGGTGGTGGCACAACTGTCTTGGTTTTAATTTGTGCTTC |
| | ATAGCCCTCCCAGGGTCCTCTCAGCCTCAAATTGCATTTCCAAATGTAGTTGAAGGACAGAGTGGGCA |
| | ACCGAAGGCAGTGGAGATGGGAAGATGAATGGCAGGGTCCTCTCCTCTCTCTCTCTGCTTCTTCAGCC |
| | TGCCTTCCACATCTCCCTTGGTGCCGCTGCTTCTCTCCGGCTTTGCACCTCTGTTCTTGAAAGGGCTG |
| | CAGAACTGGACTCAGACCACGCAAGAAGGCAAGTCCCCCTCAGCTGCCCCAGCTTCCAGCCAGCCCCA |
| | GGCTTGCCCAACGGACCACGTCCGTGAATCTGCACTGGGTGCCTGTCTTTCTCTCCCAGGAGAAGATG |
| | GGAAGATCCAGTACCCACACACAGACCCCCTTGTGTACACGCAGGAACCATAAACCAGCTGGAGGCAG |
| | CCCCTGCCCCACCCTGTCTTATCTACAAAAAATATTACAAGAGACTTTATCTCTTGATTTGCTTCATC |
| | GAGTGTCCCAACTACCTCATTTTTTTAAAATGTGAAATTAGCTTCATTTACCTTCATTGAATCCATGT |
| | TGGCGACTATTAAAAATTCCAGGCAATAAAAAGGGATGAGAGCCTGAACTAAAGCAGTGGCAATAACT |
| | GGTGAAAGAGTAAAAAAACAGATCTGATTGACTCTGGGGTGAACTGATTGACTCTCGCGTTTGACTAA |
| | ATGAGGAGGAGAGAGGGAG |
| SERPINC1 HDR | GCGGAGGAATATGTCCCAGATAGCACTGGGGGGAGTAGAGGCGGCCACGACCTGGTGAACACCTAGGA |
| insert | CGCACCATTCTCACAAAGGGAGTTTTCCACACGGACACCCCCCTCCTCACCACAGCCCTGCCAGGACG |
| sequence 2 | GGGCTGGCTACTGGCCTTATCTCACAGGTAAAACTGACGCACGGAGGAACAATATAAATTGGGGACTA |
| (SEQ ID NO: 13) | GAAAGGTGAAGAGCCAAAGTTAGAACTCAGGACCAACTTATTCTGATTTTGTTTTTCCAAACTGCTTC |
| | TCCTCTTGGGAAGTGTAAGGAAGCTGCAGCACCAGGATCAGTGAAACGCACCAGACGGCCGCGTCAGA |
| | GCAGCTCAGGTTCTGGGAGAGGGTAGCGCAGGGTGGCCACTGAGAACCGGGCAGGTCACGCATCCCCC |
| | CCTTCCCTCCCACCCCCTGCCAAGGATCCTCTCTGGCTCCATCGTAAGCAAACCTTAGAGGTTCTGGC |
| | AAGGAGAGAGATGGCTCCAGGAAATGGGGGTGTGTCACCAGATAAGGAATCTGCCTAACAGGAGGTGG |
| | GGGTTAGACCCAATATCAGGAGACTAGGAAGGAGGAGGCCTAAGGATGGGGCTTTTCTGTCACCAATC |
| | CTGTCCCTAGTGGCCCCACTGTGGGGTGGAGGGGACAGATAAAAGTACCCAGAACCAGAGCCACATTA |
| | ACCGGCCCTGGGAATATAAGGTGGTCCCAGCTCGGGGACACAGGATCCCTGGAGGCAGCAAACATGCT |
| | GTCCTGAAGTGGACATAGGGGCCCGGGTTGGAGGAAGAAGACTAGCTGAGCTCTCGGACCCCTGGAAG |
| | ATGCCATGACAGGGGGCTGGAAGAGCTAGCACAGACTAGAGAGGTAAGGGGGGTAGGGGAGCTGCCCA |
| | AATGAAAGGAGTGAGAGGTGACCCGAATCCACAGGAGAACGGGGTGTCCAGGCAAAGAAAGCAAGAGG |
| | GGTACCGCCATAGAGCCCACCGCATCCCCAGCATGCCTGCTATTGTCTTCCCAATCCTCCCCCTTGCT |
| | GTCCTGCCGGACCGTACCCCCCAGAATAGAATGACACCTACTCAGACAATGCGATGCAATTTCCTCAT |
| | TTTATTAGGAAAGGACAGTGGGAGTGGCACCTTCCAGGGTCAAGGAAGGCACGGGGGAGGGGCAAACA |
| | ACAGATGGCTGGCAACTAGAAGGCACAGTTACTTGTACAGCTCGTCCATGCCGAGAGTGATCCCGGCG |
| | GCGGTCACGAACTCCAGCAGGACCATGTGATCGCGCTTCTCGTTGGGGTCTTTGCTCAGGGCGGACTG |
| | GGTGCTCAGGTAGTGGTTGTCGGGCAGCAGCACGGGGCCGTCGCCGATGGGGGTGTTCTGCTGGTAGT |
| | GGTCGGCGAGCTGCACGCTGCCGTCCTCGATGTTGTGGCGGATCTTGAAGTTCACCTTGATGCCGTTC |
| | TTCTGCTTGTCGGCCATGATATAGACGTTGTGGCTGTTGTAGTTGTACTCAAGCTTGTGCCCCAGGAT |
| | GTTGCCGTCCTCCTTGAAGTCGATGCCCTTCAGCTCGATGCGGTTCACCAGGGTGTCGCCCTCGAACT |
| | TCACCTCGGCGCGGGTCTTGTAGTTGCCGTCGTCCTTGAAGAAGATGGTGCGCTCCTGGACGTAGCCT |
| | TCGGGCATGGCGGACTTGAAGAAGTCGTGCTGCTTCATGTGGTCGGGGTAGCGGCTGAAGCACTGCAC |
| | GCCGTAGGTCAGGGTGGTCACGAGGGTGGGCCAGGGCACGGGCAGCTTGCCGGTGGTGCAGATGAACT |
| | TCAGGGTCAGCTTGCCGTAGGTGGCATCGCCCTCGCCCTCGCCGGACACGCTGAACTTGTGGCCGTTT |
| | ACGTCGCCGTCCAGCTCGACCAGGATGGGCACCACCCCGGTGAACAGCTCCTCGCCCTTGCTAACCAT |
| | TCTAGAAGGGCCGGGATTCTCTTCCACGTCACCGCATGTCAGCAGACTGCCTCTGCCCTCGAATTCg |
| SERPINC1 HDR | CGCATTCTGTCTCCTGATCCCCCAGTAGAGTTTTGCTAAGTATTTCCCAGCTGCTCACACCCCTTAGA |
| amplicon | AACGCGCTTGGCATGCACCCCGAGGCCCTGCTCTTCTCTCCCTGTCCCACCACTTCAGGGCTGCTGGG |
| sequence 2 | GAATGGGTCTCTCTGTGGGCCACAGGTGTAACCATTGTGTTTTCCTTGTCTGTGCCAGGGACACCTTG |
| (SEQ ID NO: 14) | GCACTCAGATGCCTGAAGGTAGCAGCTTGTCCCTCTTTGCCTTCTCTAATTAGATATTCCTCTCTCCA |
| | TAAAGAAAACTATGAGAGAGGGTGGGTATGAACCAAGTTTGTTTCCTTGGTTAGTTTCCTAACCAAGT |
| | TTGAGGGTATGAACATACTCTCCTTTTCCTTTTCTATAAAGCTGAGGAGAAGAGTGAGGGAGTGTGGG |
| | CAAGAGAGGTGGCTCAGGCTTTCCCTGGGCCTGATTGAACTTTAAAACTTCTCTACTAATTAAACAAC |
| | ACTGGGCTCTACACTTTGCTTAACCCTGGGAACTGGTCATCAGCCTTTGACCTCAGTTCCCCCTCCTG |
| | ACCAGCTCTCTGCCCCACCCTGTCCTCTGGAACCTCTGCGAGATTTAGAGGAAAGAACCAGTTTTCAG |
| | GCGGATTGCCTCAGATCACACTATCTCCACTTGCCCAGCCCTGTGGAAGATTAGCGGCCATGTATTCC |
| | AATGTGATAGGAACTGTAACCTCTGGAAAAAGGTAGCGGAGGAATATGTCCCAGATAGCACTGGGGGG |
| | AGTAGAGGCGGCCACGACCTGGTGAACACCTAGGACGCACCATTCTCACAAAGGGAGTTTTCCACACG |
| | GACACCCCCCTCCTCACCACAGCCCTGCCAGGACGGGGCTGGCTACTGGCCTTATCTCACAGGTAAAA |
| | CTGACGCACGGAGGAACAATATAAATTGGGGACTAGAAAGGTGAAGAGCCAAAGTTAGAACTCAGGAC |
| | CAACTTATTCTGATTTTGTTTTTCCAAACTGCTTCTCCTCTTGGGAAGTGTAAGGAAGCTGCAGCACC |
| | AGGATCAGTGAAACGCACCAGACGGCCGCGTCAGAGCAGCTCAGGTTCTGGGAGAGGGTAGCGCAGGG |
| | TGGCCACTGAGAACCGGGCAGGTCACGCATCCCCCCCTTCCCTCCCACCCCCTGCCAAGGATCCTCTC |
| | TGGCTCCATCGTAAGCAAACCTTAGAGGTTCTGGCAAGGAGAGAGATGGCTCCAGGAAATGGGGGTGT |
| | GTCACCAGATAAGGAATCTGCCTAACAGGAGGTGGGGGTTAGACCCAATATCAGGAGACTAGGAAGGA |
| | GGAGGCCTAAGGATGGGGCTTTTCTGTCACCAATCCTGTCCCTAGTGGCCCCACTGTGGGGTGGAGGG |
| | GACAGATAAAAGTACCCAGAACCAGAGCCACATTAACCGGCCCTGGGAATATAAGGTGGTCCCAGCTC |
| | GGGGACACAGGATCCCTGGAGGCAGCAAACATGCTGTCCTGAAGTGGACATAGGGGCCCGGGTTGGAG |
| | GAAGAAGACTAGCTGAGCTCTCGGACCCCTGGAAGATGCCATGACAGGGGGCTGGAAGAGCTAGCACA |
| | GACTAGAGAGGTAAGGGGGGTAGGGGAGCTGCCCAAATGAAAGGAGTGAGAGGTGACCCGAATCCACA |
| | GGAGAACGGGGTGTCCAGGCAAAGAAAGCAAGAGGGGTACCGCCATAGAGCCCACCGCATCCCCAGCA |
| | TGCCTGCTATTGTCTTCCCAATCCTCCCCCTTGCTGTCCTGCCGGACCGTACCCCCCAGAATAGAATG |
| | ACACCTACTCAGACAATGCGATGCAATTTCCTCATTTTATTAGGAAAGGACAGTGGGAGTGGCACCTT |
| | CCAGGGTCAAGGAAGGCACGGGGGAGGGGCAAACAACAGATGGCTGGCAACTAGAAGGCACAGTTACT |
| | TGTACAGCTCGTCCATGCCGAGAGTGATCCCGGCGGCGGTCACGAACTCCAGCAGGACCATGTGATCG |
| | CGCTTCTCGTTGGGGTCTTTGCTCAGGGCGGACTGGGTGCTCAGGTAGTGGTTGTCGGGCAGCAGCAC |
| | GGGGCCGTCGCCGATGGGGGTGTTCTGCTGGTAGTGGTCGGCGAGCTGCACGCTGCCGTCCTCGATGT |
| | TGTGGCGGATCTTGAAGTTCACCTTGATGCCGTTCTTCTGCTTGTCGGCCATGATATAGACGTTGTGG |
| | CTGTTGTAGTTGTACTCAAGCTTGTGCCCCAGGATGTTGCCGTCCTCCTTGAAGTCGATGCCCTTCAG |
| | CTCGATGCGGTTCACCAGGGTGTCGCCCTCGAACTTCACCTCGGCGCGGGTCTTGTAGTTGCCGTCGT |
| | CCTTGAAGAAGATGGTGCGCTCCTGGACGTAGCCTTCGGGCATGGCGGACTTGAAGAAGTCGTGCTGC |
| | TTCATGTGGTCGGGGTAGCGGCTGAAGCACTGCACGCCGTAGGTCAGGGTGGTCACGAGGGTGGGCCA |
| | GGGCACGGGCAGCTTGCCGGTGGTGCAGATGAACTTCAGGGTCAGCTTGCCGTAGGTGGCATCGCCCT |
| | CGCCCTCGCCGGACACGCTGAACTTGTGGCCGTTTACGTCGCCGTCCAGCTCGACCAGGATGGGCACC |
| | ACCCCGGTGAACAGCTCCTCGCCCTTGCTAACCATTCTAGAAGGGCCGGGATTCTCTTCCACGTCACC |
| | GCATGTCAGCAGACTGCCTCTGCCCTCGAATTCgAGAGGGGTGAGCTTTCCCCTTGCCTGCCCCTACT |
| | GGGTTTTGTGACCTCCAAAGGACTCACAGGAATGACCTCCAACACCTTTGAGAAGACCAGGCCCTCTC |
| | CCTGGTAGTTACAGTCAAAGACCTGTTTGGAAGACGTCATTTCAAGTGCTCTCCCTCCCACCCCACCT |
| | CTTGGGGTAAGGCCTTTCCTAAGCTACCCCTTGGGTCCCTAGCCTAAGAAACAAGGGGGATGTCATCC |
| | CTGGTGTAAAGATGCTGTGCAGGAAGTCAGCACTCACGGGATCCAGGGGACGCTCCAAGGGGAATCCC |
| | CAGGGCCTGCCATCCATCCGGGAAGAGAGCAAATGCTACCCATGAGGACCTCCTCACTCCCTTTTTGC |
| | TCTTTCTTCCACTCAGATCCACCCCACTCCACCCCCACCCAAATCCCAGTGACCTTTGACTAAAGGGC |
| | CAAAACTGCTTCCTTTTCTCACAATGAGAGTTGTCCCTCCCTCAATGCCACACACACTCCCTTCTTCA |
| | TCTGAGTTGTCACAGGAGGCTAGAAACGGGGTGGTGGCACAACTGTCTTGGTTTTAATTTGTGCTTCA |
| | TAGCCCTCCCAGGGTCCTCTCAGCCTCAAATTGCATTTCCAAATGTAGTTGAAGGACAGAGTGGGCAA |
| | CCGAAGGCAGTGGAGATGGGAAGATGAATGGCAGGGTCCTCTCCTCTCTCTCTCTGCTTCTTCAGCCT |
| | GCCTTCCACATCTCCCTTGGTGCCGCTGCTTCTCTCCGGCTTTGCACCTCTGTTCTTGAAAGGGCTGC |
| | AGAACTGGACTCAGACCACGCAAGAAGGCAAGTCCCCCTCAGCTGCCCCAGCTTCCAGCCAGCCCCAG |
| | GCTTGCCCAACGGACCACGTCCGTGAATCTGCACTGGGTGCCTGTCTTTCTCTCCCAGGAGAAGATGG |
| | GAAGATCCAGTACCCACACACAGACCCCCTTGTGTACACGCAGGAACCATAAACCAGCTGGAGGCAGC |
| | CCCTGCCCCACCCTGTCTTATCTACAAAAAATATTACAAGAGACTTTATCTCTTGATTTGCTTCATCG |
| | AGTGTCCCAACTACCTCATTTTTTTAAAATGTGAAATTAGCTTCATTTACCTTCATTGAATCCATGTT |
| | GGCGACTATTAAAAATTCCAGGCAATAAAAAGGGATGAGAGCCTGAACTAAAGCAGTGGCAATAACTG |
| | GTGAAAGAGTAAAAAAACAGATCTGATTGACTCTGGGGTGAACTGATTGACTCTCGCGTTTGACTAAA |
| | TGAGGAGGAGAGAGGGAG |

### Example 2

### UMI Library Preparation and Sequencing

Input DNA (5×10³ copies) was used as the template for PCR amplification for two cycles with target specific, UMI-containing primers (Table 2) followed by a 0.5× by volume Solid Phase Reversible Immobilization purification (SPRI; Beckman Coulter, Inc.) to remove unconsumed primers. This was used as the template for a second barcoding PCR for 28-30 cycles, followed by a 0.5×SPRI purification. Samples were visualized on the Fragment Analyzer, quantified by Qubit, pooled, and sequenced using an Oxford Nanopore Technologies MinION sequencer or PacBio Sequel II instrument aiming for a coverage depth of ≥10× per UMI (100,000 reads) per sample. Sequencing adapters were added to the final barcoded libraries by ligation using kits available from the manufacturers.

TABLE 2
PCR Primers

| | | SEQ |
|---|---|---|
| Sequence Name | Sequence (5′→3′) | ID NO. |
| HBB ONT Fwd | TTTCTGTTGGTGCTGATATTGCNNNYRNNNYRNNNYRNNNAGCAGGAAGCAGA | 15 |
| | ACTCTG | |
| HBB ONT Rev 3 | ACTTGCCTGTCGCTCTATCTTCNNNYRNNNYRNNNYRNNNAGTTTAGTAGTTG | 16 |
| | GACTTAGGGAAC | |
| SC1 2kb Fwd1 ONT | TTTCTGTTGGTGCTGATATTGCNNNYRNNNYRNNNYRNNNCTCCCTCTCTCCT | 17 |
| | CCTCATTTA | |
| SC1 2kb Rev1 ONT | ACTTGCCTGTCGCTCTATCTTCNNNYRNNNYRNNNYRNNNCGCATTCTGTCTC | 18 |
| | CTGATCC | |
| TRAC UMI Karst Fwd | TTTCTGTTGGTGCTGATATTGCNNNYRNNNYRNNNYRNNNGCAGGAGGTCGGA | 19 |
| | AAGAATAA | |
| TRAC UMI Karst Rev | ACTTGCCTGTCGCTCTATCTTCNNNYRNNNYRNNNYRNNNACCCTCTCATGGC | 20 |
| | CTAGAA | |
| HBB_Karst | /5AmMC6/GCAGTCGAACATGTAGCTGACTCAGGTCACNNNYRNNNYRNNNYR | 21 |
| UMI_PacBio_F | NNNAGCAGGAAGCAGAACTCTG | |
| HBB_Karst | /5AmMC6/tggatcacttgtgcaagcatcacatcgtagNNNYRNNNYRNNNYR | 22 |
| UMI_PacBio_R | NNNAGTTTAGTAGTTGGACTTAGGGAAC | |
| SC1_Karst | /5AmMC6/GCAGTCGAACATGTAGCTGACTCAGGTCACNNNYRNNNYRNNNYR | 23 |
| UMI_PacBio_F | NNNCTCCCTCTCTCCTCCTCATTTA | |
| SC1_Karst | /5AmMC6/TGGATCACTTGTGCAAGCATCACATCGTAGNNNYRNNNYRNNNYR | 24 |
| UMI_PacBio_R | NNNCGCATTCTGTCTCCTGATCC | |
| TRAC_Karst | /5AmMC6/GCAGTCGAACATGTAGCTGACTCAGGTCACNNNYRNNNYRNNNYR | 25 |
| UMI_PacBio_F | NNNGCAGGAGGTCGGAAAGAATAA | |
| TRAC_Karst | /5AmMC6/TGGATCACTTGTGCAAGCATCACATCGTAGNNNYRNNNYRNNNYR | 26 |
| UMI_PacBio_R | NNNACCCTCTCATGGCCTAGAA | |

### Example 3

### UMI Consensus Construction and CRISPAltRations Analysis

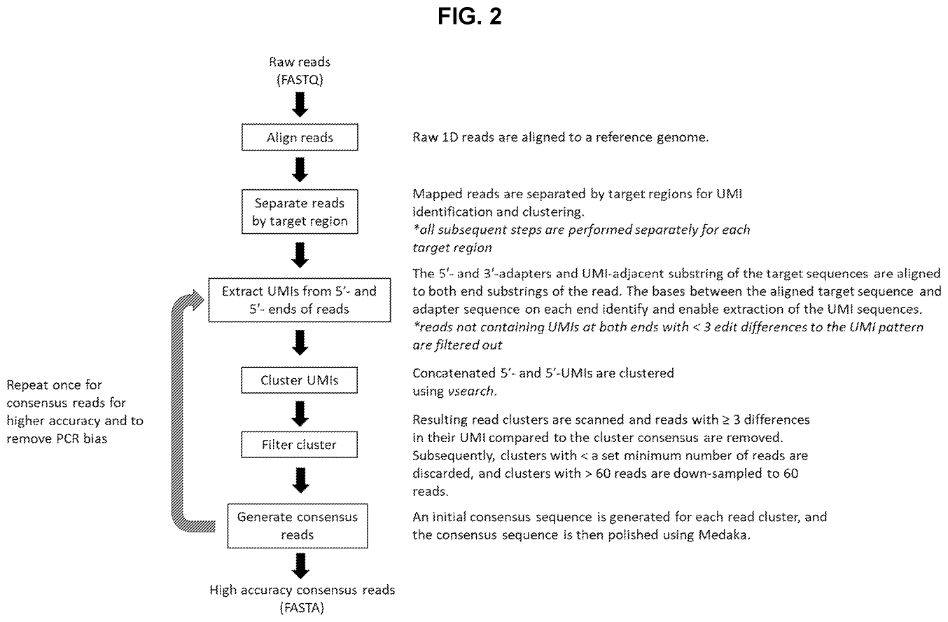

The pipeline we used for UMI identification and consensus construction was originally developed by Oxford Nanopore Technologies and made available on their github web site (github.com/nanoporetech/pipeline-umi-amplicon) as (pipeline_umi_amplicon) under Mozilla Public License version 2.0. Some improvements were made to the pipeline, but the general workflow was not changed. A critical improvement is a new UMI identification method that allows the processing of alternate UMI designs, including 18-nt structured UMIs and a random 10-nt UMI, as shown in FIG. 2.

Pipeline_umi_amplicon functions by first aligning the reads to the reference genome (hg38) using minimap2, and then the mapped reads were separated by target regions for separate UMI identification and clustering. The UMI sequences were extracted from the 5′- and 3′-ends of each read and reads not containing both UMIs are filtered out. The UMI identification step was altered to identify the UMI sequence by aligning the expected adapter and target bases surrounding the UMI to the 5′- and 3′-ends of each read, and then extracting the bases between the alignments. The reads were clustered using vsearch and the cluster consensus was generated using medaka. The process then repeats the UMI identification, clustering, and consensus construction steps on the intermediate reads for higher accuracy and to remove PCR bias that was not corrected in the first clustering and consensus step due to the higher error rate present in the UMI sequences.

The UMI pipeline generated FASTA files containing the consensus reads. These files were used as input into the CRISPAltRations pipeline for downstream analysis of CRISPR editing, including the percent HDR, percent perfect HDR, and percent imperfect HDR.

### Example 4

### UMI Consensus Construction Decreases Error Rate

After UMI consensus construction with the required UMI cluster identity set to 80% (id80) and the minimum intermediate reads per cluster set to 10 (min10), the mean error rate was calculated using an internally built error profiling tool and was decreased 16.4-fold from 8.03% to 0.491% across the three sites on the Oxford Nanopore Technologies MinION. For samples sequenced on the PacBio Sequel II with circular consensus sequencing (CCS; >3_passes/molecule) and a minimum Q score of 20 the mean error rate was decreased 23.5-fold from 0.47% to 0.017%, demonstrating that the incorporation of UMIs corrects for polymerase and/or sequencing errors (Table 3).

TABLE 3
Error Rates Before and After UMI Consensus Construction
using Oxford Nanopore Technologies and PacBio Platforms

| | | Mean error (%) | | Average of Three Sites (%) | |
|---|---|---|---|---|---|
| Platform | Target | Raw | UMI | Raw | UMI |
| ONT | HBB | 7.82 | 0.834 | 8.03 | 0.491 |
| MinION | SERPINC1 | 8.68 | 0.114 | | |
| | TRAC | 7.60 | 0.525 | | |
| PacBio | HBB | 0.17 | 0.027 | 0.47 | 0.017 |
| Sequel II | SERPINC1 | 0.18 | 0.022 | | |
| | TRAC | 1.06 | 0.013 | | |

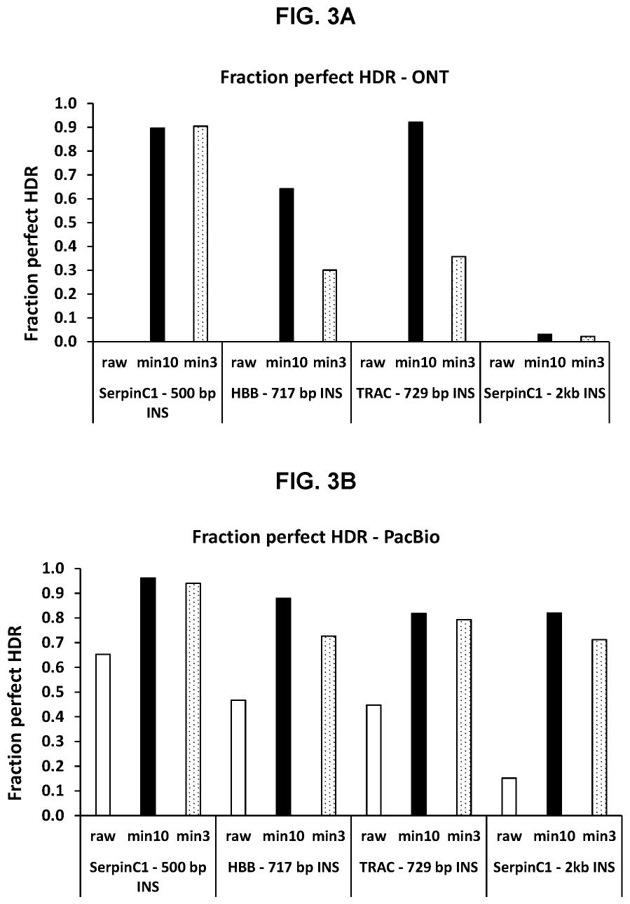

This was also reflected in the relative fraction of the total HDR reads that were considered “perfect HDR” by the CRISPAltRations pipeline. The high error rate with standard Oxford Nanopore Technologies sequencing typically results in <0.2% of HDR reads being called as perfect, where the sequence exactly matches the expected HDR event base-by-base, even with a relatively high HDR frequency (40-60%). Even with the higher accuracy of PacBio HiFi sequencing, the fraction of HDR reads that are perfect varies from 0.15-0.65 and decreases with longer HDR insertions. Universally, the fraction of HDR reads that were quantified as perfect increased after UMI consensus construction, although to a minimal degree with the longest 2 kb amplicon when sequenced using the Oxford Nanopore Technologies MinION (FIG. 3A). However, when sequenced on the PacBio Sequel II with HiFi reads, all samples were able to report >80% of the total HDR reads as perfect (FIG. 3B). The UMI min10 parameter (meaning reads were required to form a cluster) demonstrated the strongest improvement over the raw reads, compared to the min 3 parameter, for both the Oxford Nanopore Technologies and PacBio platforms. Additional UMI parameters including minimum UMI sequence identity within a cluster of 70% (id70) or 90% (id90) were investigated but did not further improve performance (data not shown). Further investigation into the minimum number of intermediate reads is planned.

### Example 5

### UMI Consensus Construction to Correct for PCR Bias

PCR bias is more pronounced as the HDR insertion size increases relative to the WT amplicon length. The longest HDR insertion in this test set is 1971 bp at the SERPINC1 locus, which is nearly equivalent to the WT amplicon length of 1991 bp. After library preparation of this sample and sequencing using the Oxford Nanopore Technologies platform the raw HDR rate decreased from the expected 31.1% HDR to 19.2%. After UMI consensus construction the total percent HDR was increased to 23.2% (min10) and 26.6% (min3), more closely matching the expected HDR frequency for this sample. Further investigation into the coverage depth requirements and ideal UMI consensus construction parameters to identify optimal sequencing and analysis conditions may improve the robustness of this methodology for error correction and PCR bias correction.

What is claimed:

1. A method for improving the accuracy of long read sequencing, the method comprising:
generating a sequencing library comprising:
(a) amplifying a locus with primers comprising a unique molecular identifier and a universal sequence to generate an initial product;
(b) purifying the initial products;
(c) amplifying the initial product with primers comprising a sequence complementary to the universal sequence and a barcode sequence to generate barcoded products;
(d) purifying the barcoded products to produce purified barcoded products;
(e) pooling the purified barcoded products to produce pooled barcoded products; and
(f) sequencing the pooled barcoded products using a long-read sequencing apparatus to generate raw nucleotide sequence data.

2. The method of claim 1, further comprising, executing on a processor:
(g) receiving raw nucleotide sequence data;
(h) aligning the raw nucleotide sequence data to a reference amplicon to generate mapped sequences;
(i) identifying and separating mapped sequences by target regions to generate a plurality of groups of target region sequences;
(j) for each group of target region sequences:
(i) analyzing the target region sequences for unique molecular identifiers and discarding target region sequences lacking a unique molecular identifier;
(ii) clustering target region sequences containing unique molecular identifiers to generate clustered target region sequences and a cluster consensus sequence;
(iii) analyzing and filtering the clustered target region sequences and discarding sequences with less than an elected number of cluster consensus sequences and downsampling clusters with greater than an elected cluster size to the elected cluster size;
(iv) generating an inital target sequence consensus sequence;
(k) repeating steps (j) on the inital target sequence consensus sequences to create a high accuracy consensus sequence for each cluster group, and correct amplification bias by clustering groups that were not similar enough to be clustered in the first round;
(l) outputting high accuracy consensus sequence data.

3. The method of claim 2, wherein step (j)(i) comprises:
aligning 5′- and 3′-adapters and UMI-adjacent substrings of the target region to both end substrings of the sequences;
nucleotides between the aligned target sequence and adapter sequence on each end identify and enable clustering of the UMI sequences; and
sequences lacking UMIs at both ends and containing less than 3 edit differences to the UMI are discarded.

4. The method of claim 2, wherein the elected number of cluster consensus sequences is between 3 and 10; and the elected cluster size is 20 to 80.

5. The method of claim 1, further comprising analyzing the raw nucleotide sequence data from claim 1(f) or the high accuracy consensus sequence data from claim 2(l), the method comprising, executing on a processor:
receiving the sequence data comprising a plurality of sequences;
analyzing and merging of the sample sequence data and outputting merged sequences;
developing target-site sequences containing predicted outcomes of repair events when a single-stranded or a double-stranded DNA oligonucleotide donor is provided and outputting the target predicted outcomes;
binning the merged sequences with the target-site sequences or the optional target predicted outcomes using a mapper and outputting target-read alignments;
re-aligning the binned target-read alignments to the target-site using an enzyme specific position-specific scoring matrix derived from biological data that is applied based on the position of a guide sequence and a canonical enzyme-specific cut site and producing a final alignment;
analyzing the final alignment and identifying and quantifying mutations within a pre-defined sequence distance window from the canonical enzyme-specific cut sites;
outputting the final alignment, analysis, and quantification results data as tables or graphics.

6. The method of claim 1, wherein purifying in steps (b) and (d) comprises solid phase reversible immobilization (SPRI) purification.

7. The method of claim 1, wherein the unique molecular identifier comprises 8-30 nucleotides.

8. The method of claim 1, wherein the unique molecular identifier comprises 8-18 nucleotides.

9. The method of claim 1, wherein the universal sequence comprises 22-30 nucleotides.

10. The method of claim 1, wherein the barcode sequence comprises 16-24 nucleotides.

11. The method of claim 1, wherein the amplifying in step (a) comprises at least 2 cycles of PCR.

12. The method of claim 1, wherein the amplifying in step (a) comprises 2-4 cycles of PCR.

13. The method of claim 1, wherein the amplifying in step (c) comprises 20-40 cycles of PCR.

14. The method of claim 1, wherein long-read sequencing apparatus are selected from Oxford Nanopore Technologies (ONT) MinION, or PacBio Sequel II.

15. The method of claim 1, wherein the sequencing error rate is reduced by at least 15-fold.


SEQUENCE LISTING (26 sequences)

SEQ ID NO: 1 - DNA, 20 bp, synthetic construct
  source 1..20: mol_type = other DNA
gagaatcaaaatcggtgaat

SEQ ID NO: 2 - DNA, 2027 bp, unidentified
  source 1..2027: mol_type = other DNA
gcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggt

SEQ ID NO: 3 - DNA, 729 bp, synthetic construct
  source 1..729: mol_type = other DNA
ggatcgggtgggactagtggcagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactggaaccggtgctggaagtggt

SEQ ID NO: 4 - DNA, 2756 bp, synthetic construct
  source 1..2756: mol_type = other DNA
gcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgggatcgggtgggactagtggcagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactggaaccggtgctggaagtggtattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggt

SEQ ID NO: 5 - DNA, 20 bp, synthetic construct
  source 1..20: mol_type = other DNA
cttgccccacagggcagtaa

SEQ ID NO: 6 - DNA, 2715 bp, synthetic construct
  source 1..2715: mol_type = other DNA
agcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaagaacaacaacaaatgaatgcatatatatgtatatgtacgtgtgtatatatacacacatatgtatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcatatcagaaagtggtggctggtgtggctaatgccctggcccacaagtatcactaagctcgctttcttgctgtccaatttctattaaaggttcctttgttccctaagtccaactactaaact

SEQ ID NO: 7 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 8 - DNA, 3432 bp, synthetic construct
  source 1..3432: mol_type = other DNA
agcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaagaacaacaacaaatgaatgcatatatatgtatatgtacgtgtgtatatatacacacatatgtatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagctgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcatatcagaaagtggtggctggtgtggctaatgccctggcccacaagtatcactaagctcgctttcttgctgtccaatttctattaaaggttcctttgttccctaagtccaactactaaact

SEQ ID NO: 9 - DNA, 20 bp, synthetic construct
  source 1..20: mol_type = other DNA
acctctggaaaaaggtaaga

SEQ ID NO: 10 - DNA, 1991 bp, synthetic construct
  source 1..1991: mol_type = other DNA
cgcattctgtctcctgatcccccagtagagttttgctaagtatttcccagctgctcacaccccttagaaacgcgcttggcatgcaccccgaggccctgctcttctctccctgtcccaccacttcagggctgctggggaatgggtctctctgtgggccacaggtgtaaccattgtgttttccttgtctgtgccagggacaccttggcactcagatgcctgaaggtagcagcttgtccctctttgccttctctaattagatattcctctctccataaagaaaactatgagagagggtgggtatgaaccaagtttgtttccttggttagtttcctaaccaagtttgagggtatgaacatactctccttttccttttctataaagctgaggagaagagtgagggagtgtgggcaagagaggtggctcaggctttccctgggcctgattgaactttaaaacttctctactaattaaacaacactgggctctacactttgcttaaccctgggaactggtcatcagcctttgacctcagttccccctcctgaccagctctctgccccaccctgtcctctggaacctctgcgagatttagaggaaagaaccagttttcaggcggattgcctcagatcacactatctccacttgcccagccctgtggaagattagcggccatgtattccaatgtgataggaactgtaacctctggaaaaaggtaagaggggtgagctttccccttgcctgcccctactgggttttgtgacctccaaaggactcacaggaatgacctccaacacctttgagaagaccaggccctctccctggtagttacagtcaaagacctgtttggaagacgtcatttcaagtgctctccctcccaccccacctcttggggtaaggcctttcctaagctaccccttgggtccctagcctaagaaacaagggggatgtcatccctggtgtaaagatgctgtgcaggaagtcagcactcacgggatccaggggacgctccaaggggaatccccagggcctgccatccatccgggaagagagcaaatgctacccatgaggacctcctcactccctttttgctctttcttccactcagatccaccccactccacccccacccaaatcccagtgacctttgactaaagggccaaaactgcttccttttctcacaatgagagttgtccctccctcaatgccacacacactcccttcttcatctgagttgtcacaggaggctagaaacggggtggtggcacaactgtcttggttttaatttgtgcttcatagccctcccagggtcctctcagcctcaaattgcatttccaaatgtagttgaaggacagagtgggcaaccgaaggcagtggagatgggaagatgaatggcagggtcctctcctctctctctctgcttcttcagcctgccttccacatctcccttggtgccgctgcttctctccggctttgcacctctgttcttgaaagggctgcagaactggactcagaccacgcaagaaggcaagtccccctcagctgccccagcttccagccagccccaggcttgcccaacggaccacgtccgtgaatctgcactgggtgcctgtctttctctcccaggagaagatgggaagatccagtacccacacacagacccccttgtgtacacgcaggaaccataaaccagctggaggcagcccctgccccaccctgtcttatctacaaaaaatattacaagagactttatctcttgatttgcttcatcgagtgtcccaactacctcatttttttaaaatgtgaaattagcttcatttaccttcattgaatccatgttggcgactattaaaaattccaggcaataaaaagggatgagagcctgaactaaagcagtggcaataactggtgaaagagtaaaaaaacagatctgattgactctggggtgaactgattgactctcgcgtttgactaaatgaggaggagagagggag

SEQ ID NO: 11 - DNA, 500 bp, synthetic construct
  source 1..500: mol_type = other DNA
cgaattcgagggcagaggcagtctgctgacatgcggtgacgtggaagagaatcccggcccttctagaatggttagcaagggcgaggagctgttcaccggggtggtgcccatcctggtcgagctggacggcgacgtaaacggccacaagttcagcgtgtccggcgagggagagggcgatgccacctacggcaagctgaccctgaagttcatctgcaccaccggcaagctgccagtgccctggcctaccctcgtgaccaccctgacctaactgtgccttctagttgccagccatctgttgtttgcgcctcactcgtgccttcattgaccctggaaggtgccactcccactgtcctttcctaataaaatgaggaaattgcatcgcattgtctgagtaggtgtcattctattctggcgtatcgagtggctcaggacagcaagagcgaggattgggaagacaatagcaggcatgctggggatgcggtgggctctatggcggtacc

SEQ ID NO: 12 - DNA, 2491 bp, synthetic construct
  source 1..2491: mol_type = other DNA
cgcattctgtctcctgatcccccagtagagttttgctaagtatttcccagctgctcacaccccttagaaacgcgcttggcatgcaccccgaggccctgctcttctctccctgtcccaccacttcagggctgctggggaatgggtctctctgtgggccacaggtgtaaccattgtgttttccttgtctgtgccagggacaccttggcactcagatgcctgaaggtagcagcttgtccctctttgccttctctaattagatattcctctctccataaagaaaactatgagagagggtgggtatgaaccaagtttgtttccttggttagtttcctaaccaagtttgagggtatgaacatactctccttttccttttctataaagctgaggagaagagtgagggagtgtgggcaagagaggtggctcaggctttccctgggcctgattgaactttaaaacttctctactaattaaacaacactgggctctacactttgcttaaccctgggaactggtcatcagcctttgacctcagttccccctcctgaccagctctctgccccaccctgtcctctggaacctctgcgagatttagaggaaagaaccagttttcaggcggattgcctcagatcacactatctccacttgcccagccctgtggaagattagcggccatgtattccaatgtgataggaactgtaacctctggaaaaaggtacgaattcgagggcagaggcagtctgctgacatgcggtgacgtggaagagaatcccggcccttctagaatggttagcaagggcgaggagctgttcaccggggtggtgcccatcctggtcgagctggacggcgacgtaaacggccacaagttcagcgtgtccggcgagggagagggcgatgccacctacggcaagctgaccctgaagttcatctgcaccaccggcaagctgccagtgccctggcctaccctcgtgaccaccctgacctaactgtgccttctagttgccagccatctgttgtttgcgcctcactcgtgccttcattgaccctggaaggtgccactcccactgtcctttcctaataaaatgaggaaattgcatcgcattgtctgagtaggtgtcattctattctggcgtatcgagtggctcaggacagcaagagcgaggattgggaagacaatagcaggcatgctggggatgcggtgggctctatggcggtaccagaggggtgagctttccccttgcctgcccctactgggttttgtgacctccaaaggactcacaggaatgacctccaacacctttgagaagaccaggccctctccctggtagttacagtcaaagacctgtttggaagacgtcatttcaagtgctctccctcccaccccacctcttggggtaaggcctttcctaagctaccccttgggtccctagcctaagaaacaagggggatgtcatccctggtgtaaagatgctgtgcaggaagtcagcactcacgggatccaggggacgctccaaggggaatccccagggcctgccatccatccgggaagagagcaaatgctacccatgaggacctcctcactccctttttgctctttcttccactcagatccaccccactccacccccacccaaatcccagtgacctttgactaaagggccaaaactgcttccttttctcacaatgagagttgtccctccctcaatgccacacacactcccttcttcatctgagttgtcacaggaggctagaaacggggtggtggcacaactgtcttggttttaatttgtgcttcatagccctcccagggtcctctcagcctcaaattgcatttccaaatgtagttgaaggacagagtgggcaaccgaaggcagtggagatgggaagatgaatggcagggtcctctcctctctctctctgcttcttcagcctgccttccacatctcccttggtgccgctgcttctctccggctttgcacctctgttcttgaaagggctgcagaactggactcagaccacgcaagaaggcaagtccccctcagctgccccagcttccagccagccccaggcttgcccaacggaccacgtccgtgaatctgcactgggtgcctgtctttctctcccaggagaagatgggaagatccagtacccacacacagacccccttgtgtacacgcaggaaccataaaccagctggaggcagcccctgccccaccctgtcttatctacaaaaaatattacaagagactttatctcttgatttgcttcatcgagtgtcccaactacctcatttttttaaaatgtgaaattagcttcatttaccttcattgaatccatgttggcgactattaaaaattccaggcaataaaaagggatgagagcctgaactaaagcagtggcaataactggtgaaagagtaaaaaaacagatctgattgactctggggtgaactgattgactctcgcgtttgactaaatgaggaggagagagggag

SEQ ID NO: 13 - DNA, 1971 bp, synthetic construct
  source 1..1971: mol_type = other DNA
gcggaggaatatgtcccagatagcactggggggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtccctagtggccccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggggtaccgccatagagcccaccgcatccccagcatgcctgctattgtcttcccaatcctcccccttgctgtcctgccggaccgtaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactcaagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctaaccattctagaagggccgggattctcttccacgtcaccgcatgtcagcagactgcctctgccctcgaattcg

SEQ ID NO: 14 - DNA, 3962 bp, synthetic construct
  source 1..3962: mol_type = other DNA
cgcattctgtctcctgatcccccagtagagttttgctaagtatttcccagctgctcacaccccttagaaacgcgcttggcatgcaccccgaggccctgctcttctctccctgtcccaccacttcagggctgctggggaatgggtctctctgtgggccacaggtgtaaccattgtgttttccttgtctgtgccagggacaccttggcactcagatgcctgaaggtagcagcttgtccctctttgccttctctaattagatattcctctctccataaagaaaactatgagagagggtgggtatgaaccaagtttgtttccttggttagtttcctaaccaagtttgagggtatgaacatactctccttttccttttctataaagctgaggagaagagtgagggagtgtgggcaagagaggtggctcaggctttccctgggcctgattgaactttaaaacttctctactaattaaacaacactgggctctacactttgcttaaccctgggaactggtcatcagcctttgacctcagttccccctcctgaccagctctctgccccaccctgtcctctggaacctctgcgagatttagaggaaagaaccagttttcaggcggattgcctcagatcacactatctccacttgcccagccctgtggaagattagcggccatgtattccaatgtgataggaactgtaacctctggaaaaaggtagcggaggaatatgtcccagatagcactggggggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtccctagtggccccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggggtaccgccatagagcccaccgcatccccagcatgcctgctattgtcttcccaatcctcccccttgctgtcctgccggaccgtaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactcaagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctaaccattctagaagggccgggattctcttccacgtcaccgcatgtcagcagactgcctctgccctcgaattcgagaggggtgagctttccccttgcctgcccctactgggttttgtgacctccaaaggactcacaggaatgacctccaacacctttgagaagaccaggccctctccctggtagttacagtcaaagacctgtttggaagacgtcatttcaagtgctctccctcccaccccacctcttggggtaaggcctttcctaagctaccccttgggtccctagcctaagaaacaagggggatgtcatccctggtgtaaagatgctgtgcaggaagtcagcactcacgggatccaggggacgctccaaggggaatccccagggcctgccatccatccgggaagagagcaaatgctacccatgaggacctcctcactccctttttgctctttcttccactcagatccaccccactccacccccacccaaatcccagtgacctttgactaaagggccaaaactgcttccttttctcacaatgagagttgtccctccctcaatgccacacacactcccttcttcatctgagttgtcacaggaggctagaaacggggtggtggcacaactgtcttggttttaatttgtgcttcatagccctcccagggtcctctcagcctcaaattgcatttccaaatgtagttgaaggacagagtgggcaaccgaaggcagtggagatgggaagatgaatggcagggtcctctcctctctctctctgcttcttcagcctgccttccacatctcccttggtgccgctgcttctctccggctttgcacctctgttcttgaaagggctgcagaactggactcagaccacgcaagaaggcaagtccccctcagctgccccagcttccagccagccccaggcttgcccaacggaccacgtccgtgaatctgcactgggtgcctgtctttctctcccaggagaagatgggaagatccagtacccacacacagacccccttgtgtacacgcaggaaccataaaccagctggaggcagcccctgccccaccctgtcttatctacaaaaaatattacaagagactttatctcttgatttgcttcatcgagtgtcccaactacctcatttttttaaaatgtgaaattagcttcatttaccttcattgaatccatgttggcgactattaaaaattccaggcaataaaaagggatgagagcctgaactaaagcagtggcaataactggtgaaagagtaaaaaaacagatctgattgactctggggtgaactgattgactctcgcgtttgactaaatgaggaggagagagggag

SEQ ID NO: 15 - DNA, 59 bp, synthetic construct
  source 1..59: mol_type = other DNA
tttctgttggtgctgatattgcnnnyrnnnyrnnnyrnnnagcaggaagcagaactctg

SEQ ID NO: 16 - DNA, 65 bp, synthetic construct
  source 1..65: mol_type = other DNA
acttgcctgtcgctctatcttcnnnyrnnnyrnnnyrnnnagtttagtagttggacttagggaac

SEQ ID NO: 17 - DNA, 62 bp, synthetic construct
  source 1..62: mol_type = other DNA
tttctgttggtgctgatattgcnnnyrnnnyrnnnyrnnnctccctctctcctcctcattta

SEQ ID NO: 18 - DNA, 60 bp, synthetic construct
  source 1..60: mol_type = other DNA
acttgcctgtcgctctatcttcnnnyrnnnyrnnnyrnnncgcattctgtctcctgatcc

SEQ ID NO: 19 - DNA, 61 bp, synthetic construct
  source 1..61: mol_type = other DNA
tttctgttggtgctgatattgcnnnyrnnnyrnnnyrnnngcaggaggtcggaaagaataa

SEQ ID NO: 20 - DNA, 59 bp, synthetic construct
  source 1..59: mol_type = other DNA
acttgcctgtcgctctatcttcnnnyrnnnyrnnnyrnnnaccctctcatggcctagaa

SEQ ID NO: 21 - DNA, 67 bp, synthetic construct
  source 1..67: mol_type = other DNA
  misc_feature <1: note = 5'-Amino Modifier C6 modification, e.g., NH2(CH2)6PO4-
gcagtcgaacatgtagctgactcaggtcacnnnyrnnnyrnnnyrnnnagcaggaagcagaactctg

SEQ ID NO: 22 - DNA, 73 bp, synthetic construct
  source 1..73: mol_type = other DNA
  misc_feature <1: note = 5'-Amino Modifier C6 modification, e.g., NH2(CH2)6PO4-
tggatcacttgtgcaagcatcacatcgtagnnnyrnnnyrnnnyrnnnagtttagtagttggacttagggaac

SEQ ID NO: 23 - DNA, 70 bp, synthetic construct
  source 1..70: mol_type = other DNA
  misc_feature <1: note = 5'-Amino Modifier C6 modification, e.g., NH2(CH2)6PO4-
gcagtcgaacatgtagctgactcaggtcacnnnyrnnnyrnnnyrnnnctccctctctcctcctcattta

SEQ ID NO: 24 - DNA, 68 bp, synthetic construct
  source 1..68: mol_type = other DNA
  misc_feature <1: note = 5'-Amino Modifier C6 modification, e.g., NH2(CH2)6PO4-
tggatcacttgtgcaagcatcacatcgtagnnnyrnnnyrnnnyrnnncgcattctgtctcctgatcc

SEQ ID NO: 25 - DNA, 69 bp, synthetic construct
  source 1..69: mol_type = other DNA
  misc_feature <1: note = 5'-Amino Modifier C6 modification, e.g., NH2(CH2)6PO4-
gcagtcgaacatgtagctgactcaggtcacnnnyrnnnyrnnnyrnnngcaggaggtcggaaagaataa

SEQ ID NO: 26 - DNA, 67 bp, synthetic construct
  source 1..67: mol_type = other DNA
  misc_feature <1: note = 5'-Amino Modifier C6 modification, e.g., NH2(CH2)6PO4-
tggatcacttgtgcaagcatcacatcgtagnnnyrnnnyrnnnyrnnnaccctctcatggcctagaa